(19) United States
(12) Patent Application Publication
(10) Pub. No.: US 2025/0213626 A1
(43) Pub. Date: Jul. 3, 2025

(54) ALCOHOL DEGRADING COMPOSITIONS AND USES THEREOF

(71) Applicant: De Faire Medical AB, Stockholm (SE)

(72) Inventor: Johan De Faire, Stockholm (SE)

(21) Appl. No.: 18/851,739

(22) PCT Filed: Mar. 30, 2023
(86) PCT No.: PCT/EP2023/058370

§ 371 Date: Sep. 27, 2024

(30) Foreign Application Priority Data
Mar. 31, 2022 (GB) 2204716.1

(51) Int. Cl. A61K 35/742; A61K 9/48; A61K 31/198; A61K 31/721; A61K 36/899; A61P 43/00

(52) U.S. Cl. CPC A61K 35/742; A61K 9/4816; A61K 9/485; A61K 9/4858; A61K 31/198; A61K 31/721; A61K 36/899; A61P 43/00

(57) ABSTRACT

The present invention provides an alcohol degrading composition comprising one or more bacterial species of the Bacillus genus, rice bran, L-cysteine and a high molecular weight low osmolality carbohydrate. Methods of using the composition are also provided herein.

The present invention provides an alcohol degrading composition comprising one or more bacterial species of the Bacillus genus, rice bran, L-cysteine and a high molecular weight low osmolality carbohydrate. Methods of using the composition are also provided herein.

### BACKGROUND

The fermentation of sugar into ethanol is one of the earliest biotechnologies employed by humans. The intoxicating effects of ethanol consumption have been known since ancient times. Ethanol has been used by humans since prehistory as the intoxicating ingredient of alcoholic beverages. Dried residue on 9,000-year-old pottery found in China suggests that Neolithic people consumed alcoholic beverages. Today, social drinking is tolerated in many cultures around the world. It is accepted as a legitimate way to celebrate special occasions or just to relax after a hard day at work. Drinking in moderation tends to be viewed as a harmless activity.

There are a number of benefits that people obtain from social drinking. This is why the activity has been popular for thousands of years. Alcohol is often described as a social lubricant. People tend to feel more relaxed after a drink or two and a bit less self-conscious. There are many social occasions that are based around alcohol consumption. Some studies even suggest that drinking in moderation may bring certain health benefits. It is these beneficial aspects of alcohol that ensure its continued popularity.

However, even those people who drink in moderation can encounter alcohol related problems. Alcohol is a toxin that can cause damage to the body even in small doses. Those who drink regularly above the safe limits are at increased risk of health problems, including but not limited to certain cancers, cardiovascular events, high blood pressure, accidents while under the influence, and progression to alcohol abuse and addiction. For some individuals, it is not safe for them to drink any alcohol at all.

Alcohol intoxication, also known as drunkenness or alcohol poisoning, is the negative behavior and physical effects that occur as a consequence of recent alcohol consumption. Symptoms at lower doses may include mild sedation and poor coordination. At higher doses, there may be slurred speech, trouble walking, and vomiting. Extreme doses may result in a respiratory depression, coma, or death. Complications may include seizures, aspiration pneumonia, injuries including suicide, and low blood sugar.

Alcohol is mostly metabolized in the liver, which is why the liver is particularly at risk of damage. Drinking heavily significantly increases the risk of alcoholic fatty liver, an early and reversible consequence of excessive alcohol intake. Chronic drinking alters the liver's metabolism of fats, and excess fat accumulates in the liver. Other effects on the liver include long-term inflammation (alcoholic hepatitis). This can lead to scar tissue and finally liver cirrhosis.

There is a need for a novel composition, supplement and/or therapeutic that reduces the undesirable effects associated with alcohol ingestion.

### BRIEF SUMMARY OF THE DISCLOSURE

The invention is based on the surprising finding that L-cysteine can be used to increase alcohol degradation by one or more bacterial species of the Bacillus genus. The inventors have surprisingly discovered that L-cysteine can be used to increase alcohol degradation by one or more bacterial species of the Bacillus genus particularly when combined with a high molecular weight low osmolality carbohydrate, such as dextrin. The inventors have shown that L-cysteine can be used to increase alcohol degradation by one or more bacterial species of the Bacillus genus in the gut, particularly when combined with a high molecular weight low osmolality carbohydrate, such as dextrin. Advantageously, the inventors have developed a novel composition to reduce uptake of alcohol into the blood and, therefore, to reduce the undesirable effects associated with alcohol ingestion.

The inventors have surprisingly shown that the compositions described herein accelerate the body's processing of alcohol by accelerating alcohol degradation, particularly in the gut. Generally, about 80% of alcohol that is ingested resides in the small intestine before being absorbed to the blood. The compositions described herein advantageously reduce the amount of alcohol absorption from the gut into the blood (and thus reduce the amount of alcohol that is processed by the liver). The inventors have shown that uptake of the compositions described herein prior to alcohol ingestion significantly reduces blood alcohol levels and breath alcohol levels after alcohol ingestion, as compared to placebo.

Accordingly, in one aspect an alcohol degrading composition comprising one or more bacterial species of the Bacillus genus, rice bran, L-cysteine and a high molecular weight low osmolality carbohydrate is provided.

It is suggested that a high molecular weight low osmolality carbohydrate (e.g. dextrin) and/or L-cysteine create a micro-environment in the gut that makes the microbial consortium of the compositions provided herein, upon resuscitation in the intestinal tract, excrete an enzyme cascade targeted towards short carbon chains, e.g. ethanol/alcohol, resulting in preferential targeting of these substrates. The targeted enzymes preferentially act on the alcohol residing in the intestinal tract and break it down into carbon dioxide and water, bypassing the liver process of converting alcohol into acetaldehyde and acetic acid/acetate (which are hangover metabolites that would otherwise be formed by the liver's conversion of alcohol). The compositions described herein thus accelerate the body's processing of alcohol (away from the liver).

Upon resuscitation in the intestinal tract, the Bacillus spp. of the composition (and their endospores) scan the biochemical conditions of their micro-environment and start to excrete a unique selection of bio-active substances to optimise the conditions, e.g. pH, conductivity, electrolytes, for their survival and multiplication. Nutrients and substrates are essential for survival and subsequent multiplication. When alcohol/ethanol/ethyl alcohol is present in the micro-environment, alcohol-targeted enzymes are excreted to break down alcohol into fragments containing carbon. Carbon that cannot be used by the microbes, or neighbouring tissue cells, as a nutrient will be biochemically metabolised into water and carbon dioxide that can escape the body system without causing any biological consequences/symptoms. The high molecular weight low osmolality carbohydrate (e.g. dextrin) and/or L-cysteine provided in the compositions described herein enhance this environmental scan and selective excretion of bio-active substances for optimised enzymatic conditions.

Advantageously, the compositions provided herein may be formulated as an acid resistant tablet or capsule. Such formulations are known to resist the acid in the stomach, only to dissolve once reaching the duodenum. The Bacillus spp. of the composition can then be released to settle in the upper part of the intestinal tract where they can reside for about one day before being eliminated from the body through the feces. The bacterial spp. of the composition described herein were selected to preferably and effectively metabolize ethyl alcohol into CO₂ and water, thus reducing the further resorption of alcohol from the intestinal tract. As a consequence, less alcohol is expected to be absorbed by the body, and damage of organs through alcohol degradation products is expected to be diminished.

As discussed above, in one aspect, the invention provides an alcohol degrading composition comprising one or more bacterial species of the Bacillus genus, rice bran, L-cysteine and a high molecular weight low osmolality carbohydrate.

Suitably, the one or more bacterial species of the Bacillus genus may be selected from B. subtilis and B. coagulans.

Suitably, the composition may comprise B. subtilis and B. coagulans.

### Suitably,

a) the B. subtilis species may be selected from the group consisting of: Bacillus subtilis strain DFM 0326 (LMG P-32899) and Bacillus subtilis strain DFM 1015 (LMG P-32900); and/or c) the B. coagulans species may be Bacillus coagulans strain DFM 0705 (LMG P-32921).

Suitably, the high molecular weight low osmolality carbohydrate may be dextrin.

Suitably, the alcohol may be ethyl alcohol (ethanol).

Suitably, the composition may further comprise one or more bacterial species selected from the group consisting of: Bacillus amyloliquefaciens, Bacillus velezensis, Bacillus sp MT 03, Bacillus atrophaeus, and Pediococcus pentosaceus.

Suitably, the composition may comprise at least about 10% w/w of L-cysteine.

Suitably, the composition may comprise at least about 10,000 cfu/g of bacteria of the Bacillus genus.

Suitably, the composition may comprise at least about 67% w/w of rice bran.

Suitably, the composition may comprise at least about 0.5% w/w of high molecular weight low osmolality carbohydrate.

Suitably, the composition may further comprise one or more of: vitamin B12, a fatty acid magnesium salt, calcium phosphate, potassium phosphate, silicon dioxide and cellulose, optionally wherein the fatty acid magnesium salt is magnesium stearate.

Suitably, the composition may be formulated as an acid resistant tablet or capsule. Suitably, the acid resistant tablet or capsule may comprise a film coating, wherein the film coating comprises hydroxypropyl methylcellulose (HPMC).

Suitably, in some examples, the one or more bacterial species of the Bacillus genus is not genetically modified.

The invention also provides the use of an alcohol degrading composition as described herein, for degrading alcohol.

The invention further provides the use of L-cysteine for increasing alcohol degradation by one or more bacterial species of the Bacillus genus.

Suitably, the L-cysteine may be combined with a high molecular weight low osmolality carbohydrate, optionally wherein the high molecular weight low osmolality carbohydrate is dextrin.

Suitably, the L-cysteine may be combined with rice bran.

Suitably, the one or more bacterial species of the Bacillus genus may be selected from B. subtilis and B. coagulans, optionally wherein the composition comprises B. subtilis and B. coagulans.

### Suitably,

a) the B. subtilis species may be selected from the group consisting of: Bacillus subtilis strain DFM 0326 (LMG P-32899) and Bacillus subtilis strain DFM 1015 (LMG P-32900); and/or c) the B. coagulans species may be Bacillus coagulans strain DFM 0705 (LMG P-32921).

Suitably, the alcohol may be ethyl alcohol (ethanol).

Suitably, the use may be for degrading alcohol in a subject. Suitably, the use may be for metabolising alcohol in the gut of the subject, optionally wherein the use is for metabolising alcohol in the intestine of the subject, more optionally wherein the use is for metabolising alcohol in the small intestine of the subject.

Suitably, the use may be for reducing absorption of alcohol into the blood of the subject.

Suitably, the use may be for reducing breath or blood alcohol concentration in the subject.

The invention further provides a composition as described herein for use as a medicament.

The invention further provides a composition described herein for use in degrading alcohol in a subject.

A composition described herein for use in preventing and/or treating alcohol-induced organ damage in a subject is also provided. Suitably, the organ may be the liver and/or the pancreas.

Suitably, the composition may be for use in preventing and/or treating a disease, condition or illness selected from the group consisting of: alcohol induced fatty liver, alcohol induced hepatitis, liver cirrhosis, alcohol induced cancer, cardio-vascular conditions, obesity, neuropathy, neurodegenerative diseases, hangover symptoms, flushing syndrome, headache and/or intoxication by acetaldehyde. Suitably, the cancer may be selected from: liver cancer, pancreatic cancer, breast cancer, esophageal cancer and oropharyngolaryngeal cancer.

Suitably, the composition may be for administration before alcohol ingestion.

A method of degrading alcohol in a subject, comprising administering a composition described herein to the subject is provided.

The invention further provides a method of preventing and/or treating alcohol-induced organ damage in a subject, comprising administering a composition described herein to the subject. Suitably, the organ may be the liver and/or the pancreas.

Suitably, the method may be for preventing and/or treating a disease, condition or illness selected from the group consisting of: alcohol induced fatty liver, alcohol induced hepatitis, liver cirrhosis, alcohol induced cancer, cardio-vascular conditions, obesity, neuropathy, neurodegenerative diseases, hangover symptoms, flushing syndrome, headache and/or intoxication by acetaldehyde. Suitably, the cancer may be selected from: liver cancer, pancreatic cancer, breast cancer, esophageal cancer and oropharyngolaryngeal cancer.

Suitably, the composition may be administered before alcohol ingestion.

The present invention provides an alcohol degrading composition comprising one or more bacterial species of the Bacillus genus, rice bran, L-cysteine and a high molecular weight low osmolality carbohydrate. Methods of using the composition are also provided herein.

In each of the aspects and embodiments of the invention described herein, rice bran may be replaced with any suitable cereal (e.g. cereal grain) unless the context provides otherwise. In such aspects and embodiments, a suitable cereal may be any suitable cereal bran. Suitable cereals and suitable cereal brans are discussed elsewhere herein.

Throughout the description and claims of this specification, the words “comprise” and “contain” and variations of them mean “including but not limited to”, and they are not intended to (and do not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

Various aspects of the invention are described in further detail below.

### Budapest Treaty on the International Recognition of the Deposit of Microorganisms for the Purpose of Patent Procedures

Several strains of bacillus species described in this application were deposited with the Belgian Coordinated Collections of Micro-organisms (BCCM) Laboratorium voor Microbiologie—Bacteriēnverzameling (LMG) which is an International Depositary Authority located at Universiteit Gent, K. L. Ledeganckstraat 35, 9000 Gent, Belgium. The deposits were made under the terms of the Budapest Treaty on the International Recognition of the Deposit of Microorganisms for the Purposes of Patent Procedure.

### BRIEF DESCRIPTION OF THE FIGURES

Embodiments of the invention are further described hereinafter with reference to the accompanying drawings, in which:

FIG. 1 shows mean blood alcohol concentrations after uptake of 0.3 g/kg body weight. (n=24).

FIG. 2 shows mean reduction of alcohol absorption into the blood by AB001.

FIG. 3 shows mean breath alcohol concentrations after uptake of 0.3 g/kg body weight. (n=24).

FIG. 4 shows mean reduction in breath alcohol levels by AB001.

FIG. 5 shows mean blood alcohol concentrations after uptake of 2×0.3 g/kg body weight with a light breakfast in between (30 min, n=24).

FIG. 6 shows mean reduction of alcohol absorption into the blood by AB001.

FIG. 7 shows mean breath alcohol concentrations after uptake of 2×0.3 g/kg body weight with a light breakfast in between (30 min, n=24).

FIG. 8 shows data generated from individuals (74 men and women age 26 to 66) having ingested 60 cc of wine (14%) consumed in 1 h. The individuals were given the following composition at 600 mg/dose: Fermented rice bran 414 mg, L-cysteine 120 mg, dextrin 10 mg, Vitamin B12 0.90 mcg, Excipients 26 mg, HPMC capsules.

FIG. 9 shows data generated from individuals (84 men, 56, and women, 28, age 26 to 72) having ingested 60 cc of wine (14%) consumed in 1 h. The individuals were given the following composition: Fermented rice bran 69%, L-cysteine 20%, dextrin 2%, Vitamin B12 0.00002%, Excipients 9%, HPMC capsules, at the indicated dosages.

FIG. 10 shows data generated from individuals (87 men and women age 26 to 66) having ingested 60 cc of wine (14%) consumed in 1 h. The individuals were given the following composition, at 750 mg/dose: Fermented rice bran 518 mg, L-cysteine 150 mg, dextrin 15 mg, Vitamin B112 0.90 mcg, Excipients 67 mg, HPMC capsules, and response time was determined.

FIG. 11 shows data generated from individuals (62 men and women age 26 to 66) having ingested 60 cc of wine (14%) consumed in 1 h. The individuals were given the following Composition: Fermented rice bran 69%, L-cysteine 20%, dextrin 2%, Vitamin B12 0.00002%, Excipients 9%, HPMC capsules. Time-cut in % to 0.05‰ compared to “predicted time to soberness” according to iBAC manual (acc. to sc.edu* (*https://sc.edu/about/offices_and_divisions/fraternity_and_sorority_life/documents/bac-charts1617.pdf).

FIG. 12 shows changes in bilirubin content in plasma; data generated from an in vivo alcohol study in mice. IC=Intact Control group with standard rodent fed, CMD=Placebo Group, feed with 10% ethanol and maltodextrin mixed with hight-fat and hight-carbo diet fed (Western Diet). PB=Probiotic Group, feed with 10% ethanol and AB001 mixed with hight-fat and hight-carbo diet fed (Western Diet).

FIG. 13 shows body weight change during the in vivo alcohol study in mice.

FIG. 14 provides an overview of the experimental design for the in vivo alcohol study in mice.

The patent, scientific and technical literature referred to herein establish knowledge that was available to those skilled in the art at the time of filing. The entire disclosures of the issued patents, published and pending patent applications, and other publications that are cited herein are hereby incorporated by reference to the same extent as if each was specifically and individually indicated to be incorporated by reference. In the case of any inconsistencies, the present disclosure will prevail.

Various aspects of the invention are described in further detail below.

### DETAILED DESCRIPTION

The invention is based on the surprising finding that L-cysteine can be used to increase alcohol degradation by one or more bacterial species of the Bacillus genus. The inventors have surprisingly discovered that L-cysteine can increase alcohol degradation by one or more bacterial species of the Bacillus genus particularly when combined with a high molecular weight low osmolality carbohydrate, such as dextrin. The data presented herein demonstrates that L-cysteine, particularly when combined with a high molecular weight low osmolality carbohydrate, such as dextrin, can be used to increase alcohol degradation by one or more bacterial species of the Bacillus genus in the gut. Advantageously, the inventors have developed a novel composition to reduce uptake of alcohol into the blood and, therefore, to reduce the undesirable effects associated with alcohol ingestion.

Accordingly, an alcohol degrading composition comprising one or more bacterial species of the Bacillus genus, rice bran, L-cysteine and a high molecular weight low osmolality carbohydrate is provided herein.

The composition provided herein may be referred to as “MYRKL” and/or “AB001”. Additionally, the composition provided herein may be referred to as “Pinch” unless the context defines “Pinch” otherwise (see Example 1).

As used herein “alcohol” refers to any of a class of organic compounds characterized by one or more hydroxyl (—OH) groups attached to a carbon atom of an alkyl group (hydrocarbon chain). Alcohols may be considered as organic derivatives of water (H₂O) in which one of the hydrogen atoms has been replaced by an alkyl group, typically represented by R in organic structures. For example, in ethanol (or ethyl alcohol) the alkyl group is the ethyl group, —CH₂CH₃. Alcohols may be classified as primary, secondary, or tertiary, according to which carbon of the alkyl group is bonded to the hydroxyl group. Most alcohols are colourless liquids or solids at room temperature. Alcohols of low molecular weight are highly soluble in water; with increasing molecular weight, they become less soluble in water, and their boiling points, vapour pressures, densities, and viscosities increase. A person skilled in the art would readily be able to identify an alcohol using methods routine in the art. Non-limiting examples of alcohol include methanol and ethanol.

As would be clear to the skilled person, an “alcohol degrading composition”, as referred to herein, is a composition which degrades alcohol. In other words, an “alcohol degrading composition”, as used herein, is a composition which breaks down (e.g. biochemically breaks down) alcohol. In the context of the invention, degradation (e.g. breakdown) of a substance (such as alcohol) involves converting the substance into one or more other distinct substances. Furthermore, as would be understood by the person skilled in the art, “alcohol degrading”, as used herein, refers to the breaking down of alcohol and, “alcohol degradation”, as used herein, refers to the breakdown of alcohol. As would be clear to the skilled person, the composition described herein can be used in vitro or in vivo.

Alcohol may be degraded (e.g. broken down) in several different ways. Particularly relevant in the context of the present invention is biochemical degradation. Accordingly, in one example, alcohol may be degraded (e.g. broken down) biochemically. As would be known to the skilled person, the biochemical break down of a substance may involve enzyme catalyzed reactions. Thus, in one example, alcohol may be degraded enzymatically.

In the body, alcohol is metabolized by several processes or pathways. The most common of these pathways involves two enzymes—alcohol dehydrogenase (ADH) and aldehyde dehydrogenase (ALDH). These enzymes help break apart the alcohol molecule, making it possible to eliminate it from the body. Most ethanol in the body following alcohol ingestion is broken down in the liver by ADH. First, ADH metabolizes alcohol to acetaldehyde, a highly toxic substance and known carcinogen. Then, in a second step, acetaldehyde is further metabolized by acetaldehyde dehydrogenase to another, less active by product called acetate, which then is broken down into water and carbon dioxide for easy elimination. Acetate is broken down to carbon dioxide and water mainly in tissues other than the liver. Alcohol-dehydrogenase generates free oxygen radicals that affect the genes. The enzymes cytochrome P450 2E1 (CYP2E1) and catalase also break down alcohol to acetaldehyde. However, CYP2E1 only is active after a person has consumed large amounts of alcohol, and catalase metabolizes only a small fraction of alcohol in the body. Small amounts of alcohol also are removed by interacting with fatty acids to form compounds called fatty acid ethyl esters (FAEEs). These compounds have been shown to contribute to damage to the liver and pancreas.

Advantageously, the compositions described herein promote the degradation (e.g. break down) of alcohol into carbon dioxide and water in the gut, whilst by-passing the liver's conversion of alcohol into noxious metabolites, including acetaldehyde and in turn acetic acid, which are considered as hangover metabolites that are formed by the liver's conversion of alcohol. The compositions described herein may promote the degradation of alcohol into carbon dioxide and water in the gut via intermediary metabolites that are not acetaldehyde and/or acetic acid.

“Metabolism”, as used herein, is a term used to describe all biochemical reactions involved in maintaining the living state of cells and organisms. For example, metabolism includes all the biochemical reactions involved in converting one molecule into another (to essentially maintain the living state of a cell or an organism). Metabolism includes processes for cell growth, reproduction, response to the environment, survival mechanisms, sustenance, and maintenance of cell structure and integrity. The biochemical reactions involved in metabolism utilize various enzymes.

In one example, alcohol may be metabolized (i.e. alcohol may be degraded via metabolism). Accordingly, in one example, an alcohol metabolizing composition comprising one or more bacterial species of the Bacillus genus, rice bran, L-cysteine and a high molecular weight low osmolality carbohydrate (e.g. dextrin) is provided. As is known to the skilled person, metabolism may be enzymatic thus, in some examples, alcohol may be metabolized enzymatically.

Metabolism may be categorized into two: catabolism and anabolism. Catabolism includes a series of degradative biochemical reactions that break down complex molecules into smaller units, usually releasing energy in the process. For example, catabolism may be used to refer to all biochemical or enzymatic reactions involved in the breakdown of organic or inorganic materials such as proteins, sugars, fatty acids, etc. Anabolism includes a sequence of biochemical reactions that constructs or synthesizes molecules from smaller units, usually requiring the input of energy (ATP) in the process. Catabolism thus refers to destructive biochemical reactions which occur in an organism whereas metabolism refers to the whole set of biochemical reactions in the organism, which can be either constructive or destructive.

In some examples, the degradation of alcohol via metabolism may be considered as catabolism. Accordingly, in some examples, alcohol may be catabolized (i.e alcohol may be degraded via catabolism). In a further example, alcohol may be catabolized enzymatically.

In some examples, alcohol is degraded in a subject. By way of non-limiting example, alcohol may be broken down enzymatically, metabolized or catabolized in a subject. Where alcohol degradation occurs in a subject (e.g. particularly in the duodenum and/or small intestine) this may be referred to digestion. In some examples, alcohol may be degraded by biochemical digestion.

In one example, the alcohol is ethyl alcohol (also known as ethanol).

Ethanol is an organic chemical compound. It is a simple alcohol with the chemical formula C₂H₆O. Its formula can be also written as CH₃—CH₂—OH or C₂H₅OH (an ethyl group linked to a hydroxyl group), and is often abbreviated as EtOH. Ethanol is a volatile, flammable, colorless liquid with a characteristic wine-like odor and pungent taste. It is a psychoactive drug, recreational drug, and the active ingredient in alcoholic drinks. Accordingly, in one example, the alcohol is comprised within an alcoholic drink. Non-limiting examples of alcoholic drinks include beer, wine and spirits (e.g. vodka).

Accordingly, in one example, an ethanol degrading composition comprising one or more bacterial species of the Bacillus genus, rice bran, L-cysteine and a high molecular weight low osmolality carbohydrate (e.g. dextrin) is provided herein.

In another example, an ethanol metabolizing composition comprising one or more bacterial species of the Bacillus genus, rice bran, L-cysteine and a high molecular weight low osmolality carbohydrate (e.g. dextrin) is provided herein.

As discussed elsewhere herein, the alcohol degrading composition provided herein comprises one or more bacterial species of the Bacillus genus. For example, the composition may comprise two or more bacterial species of the Bacillus genus. In another example, the composition may comprise three or more bacterial species of the Bacillus genus. In a further example, the composition may comprise four or more bacterial species of the Bacillus genus. In another example, the composition may comprise five or more bacterial species of the Bacillus genus. In a further example, the composition may comprise six or more bacterial species of the Bacillus genus. In another example, the composition may comprise seven or more bacterial species of the Bacillus genus.

As is known in the art, a genus is made up of several species. The genus “Bacillus” thus includes all species within the genus “Bacillus,” known to those of skill in the art, including but not limited to Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, Bacillus velezensis, Bacillus sp MT 03, Bacillus atrophaeus, and Bacillus thuringiensis. It is recognized that the genus Bacillus continues to undergo taxonomical reorganization. Thus, it is intended that the genus include species that have been reclassified, including but not limited to such organisms as B. stearothermophilus, which is now named “Geobacillus stearothermophilus.” The production of resistant endospores in the presence of oxygen is considered the defining feature of the genus Bacillus, although this characteristic also applies to the recently named Alicyclobacillus, Amphibacillus, Aneurinibacillus, Anoxybacillus, Brevibacillus, Filobacillus, Gracilibacillus, Halobacillus, Paenibacillus, Salibacillus, Thermobacillus, Ureibacillus, and Virgibacillus.

Species within the genus Bacillus are gram-positive bacteria classified as members of the Family Bacillaceae, Order Bacillales, Class Bacilli. As used herein, “Bacillus species” (“Bacillus sp.”) refers to a species within the genus “Bacillus”.

Bacillus species found to be particularly important in the context of the present invention include Bacillus subtilis (B. subtilis) and Bacillus coagulans (B. coagulans). As described in the examples below, these bacteria are abundant in the composition of the invention and are particularly effective at degrading (e.g. metabolizing) ethyl alcohol into carbon dioxide and water, especially within the gut.

Accordingly, in one example, the one or more bacterial species of the Bacillus genus is selected from B. subtilis and B. coagulans.

In another example, the composition provided herein comprises B. subtilis and B. coagulans.

Any suitable B. subtilis strains and/or B. coagulans strains may be used in accordance with the present invention. A person skilled in the art would readily be able to identify suitable strains. Bacillus subtilis strain DFM 0326 (LMG P-32899), Bacillus subtilis strain DFM 1015 (LMG P-32900) and Bacillus coagulans strain DFM 0705 (LMG P-32921) have been found to be particularly important in the context of the present invention.

A Bacillus subtilis strain deposited under LMG-P accession number 32899 may be referred to herein as “DFM 0326” or “strain DFM 0326”. Bacillus subtilis strain DFM 0326 (deposited under LMG-P accession number 32899) was deposited at the Belgian Coordinated Collections of Micro-organisms (BCCM), Laboratorium voor Microbiologie—Bacteriēnverzameling (LMG), Universiteit Gent, K. L. Ledeganckstraat 35, 9000 Gent, Belgium under The Budapest Treaty of 1977 on 22 Nov. 2022.

A Bacillus subtilis strain deposited under LMG-P accession number 32900 may be referred to herein as “DFM 1015” or “strain DFM 1015”. Bacillus subtilis strain DFM 1015 (deposited under LMG-P accession number 32900) was deposited at the Belgian Coordinated Collections of Micro-organisms (BCCM), Laboratorium voor Microbiologie—Bacteriēnverzameling (LMG), Universiteit Gent, K. L. Ledeganckstraat 35, 9000 Gent, Belgium under The Budapest Treaty of 1977 on 22 Nov. 2022.

A Bacillus coagulans strain deposited under LMG-P accession number 32921 may be referred to herein as “DFM 0705” or “strain DFM 0705”. Bacillus coagulans strain DFM 0705 (deposited under LMG-P accession number 32921) was deposited at the Belgian Coordinated Collections of Micro-organisms (BCCM), Laboratorium voor Microbiologie—Bacteriēnverzameling (LMG), Universiteit Gent, K. L. Ledeganckstraat 35, 9000 Gent, Belgium under The Budapest Treaty of 1977 on 14 Dec. 2022.

Accordingly, in some examples the B. subtilis species may be selected from the group consisting of: Bacillus subtilis strain DFM 0326 (LMG P-32899) and Bacillus subtilis strain DFM 1015 (LMG P-32900); and/or the B. coagulans species may be Bacillus coagulans strain DFM 0705 (LMG P-32921).

Accordingly, in examples where the composition comprises B. subtilis and B. coagulans, the B. subtilis species may be Bacillus subtilis strain DFM 0326 (LMG P-32899) and the B. coagulans species may be Bacillus coagulans strain DFM 0705 (LMG P-32921).

In another example where the composition comprises B. subtilis and B. coagulans, the B. subtilis species may be Bacillus subtilis strain DFM 1015 (LMG P-32900) and the B. coagulans species may be Bacillus coagulans strain DFM 0705 (LMG P-32921).

In some examples, the composition according to the invention may comprise the B. subtilis strain DFM 0326 (LMG P-32899) and Bacillus subtilis strain DFM 1015 (LMG P-32900) in combination.

In another example where the composition comprises B. subtilis and B. coagulans, the composition may comprise the B. subtilis strain DFM 0326 (LMG P-32899) and Bacillus subtilis strain DFM 1015 (LMG P-32900) and the B. coagulans species strain DFM 0705 (LMG P-32921). Notably, the compositions used in the examples provided herein comprise the B. subtilis strain DFM 0326 (LMG P-32899) and Bacillus subtilis strain DFM 1015 (LMG P-32900) and the B. coagulans strain DFM 0705 (LMG P-32921).

Accordingly, the composition according to the present invention may comprise Bacillus subtilis strain DFM 0326 (LMG P-32899), Bacillus subtilis strain DFM 1015 (LMG P-32900) and Bacillus coagulans strain DFM 0705 (LMG P-32921).

The inventors also identified several other bacterial species that are particularly relevant in the context of the composition described herein. Accordingly, in one example, the composition may further comprise one or more bacterial species selected from the group consisting of: Bacillus amyloliquefaciens, Bacillus velezensis, Bacillus sp MT 03, Bacillus atrophaeus, and Pediococcus pentosaceus. In another example, the composition may further comprise two or more, or three or more, or four or more bacterial species selected from the group consisting of: Bacillus amyloliquefaciens, Bacillus velezensis, Bacillus sp MT 03, Bacillus atrophaeus, and Pediococcus pentosaceus.

In another example, the composition may further comprise Bacillus amyloliquefaciens, Bacillus velezensis, Bacillus sp MT 03, Bacillus atrophaeus, and Pediococcus pentosaceus.

The bacterial strains included in the alcohol degrading composition provided herein were identified to preferably and effectively metabolize ethyl alcohol into carbon dioxide and water. Advantageously, the inventors have shown that uptake of the compositions described herein prior to alcohol ingestion significantly reduces blood alcohol levels and breath alcohol levels after alcohol ingestion, as compared to placebo, thereby demonstrating that the compositions described herein reduce further absorption of alcohol from the intestinal tract following alcohol ingestion.

The composition provided herein comprises an appropriate amount or concentration of the one or more bacterial species of the Bacillus genus. The amount or concentration of the one or more bacterial species of the Bacillus genus may therefore be described by reference to the number of colony forming units (cfu) per gram of composition (cfu/g) or by the total number of colony forming units (cfu) per dose of composition (in other words the cfu per effective dose). As would be clear to a person of skill in the art, and as described in more detail elsewhere herein, a dose may include one or more dosage units (e.g. 2 dosage units). In examples wherein a plurality of dosage units are used to provide an effective dose, the cfu/dose corresponds to the total cfu over the plurality of dosage units.

In one example, the composition described herein comprises about 10,000 cfu/g of bacteria of the Bacillus genus to about 1×10⁸ cfu/g of bacteria of the Bacillus genus.

In one example, the composition described herein comprises at least about 1×10⁸ cfu/g of bacteria of the Bacillus genus. In another example, the composition described herein comprises at least about 1×10⁷ cfu/g of bacteria of the Bacillus genus. In another example, the composition described herein comprises at least about 1×10⁶ cfu/g of bacteria of the Bacillus genus. In a further example, the composition described herein comprises at least about 1×10⁵ cfu/g of bacteria of the Bacillus genus.

In one example, the composition described herein comprises at least about 10,000 cfu/g (i.e. at least about 1.0×10⁴ cfu/g) of bacteria of the Bacillus genus.

In another example, the composition described herein comprises at least about 11,000 cfu/g (i.e. at least about 1.1×10⁴ cfu/g) of bacteria of the Bacillus genus. In a further example, the composition described herein comprises at least about 12,000 cfu/g (i.e. at least about 1.2×10⁴ cfu/g) of bacteria of the Bacillus genus.

In another example, the composition described herein comprises at least about 13,000 cfu/g (i.e. at least about 1.3×10⁴ cfu/g) of bacteria of the Bacillus genus.

In another example, the composition described herein comprises at least about 14,000 cfu/g (i.e. at least about 1.4×10⁴ cfu/g) of bacteria of the Bacillus genus. In another example, the composition described herein comprises at least about 15,000 cfu/g (i.e. at least about 1.5×10⁴ cfu/g) of bacteria of the Bacillus genus.

In one example, the composition described herein comprises about 1×10⁸ cfu/g of bacteria of the Bacillus genus. In another example, the composition described herein comprises about 1×10⁷ cfu/g of bacteria of the Bacillus genus. In another example, the composition described herein comprises about 1×10⁶ cfu/g of bacteria of the Bacillus genus. In a further example, the composition described herein comprises about 1×10⁵ cfu/g of bacteria of the Bacillus genus.

In one example, the composition described herein comprises about 10,000 cfu/g (i.e. about 1.0×10⁴ cfu/g) of bacteria of the Bacillus genus.

In another example, the composition described herein comprises about 11,000 cfu/g (i.e. about 1.1×10⁴ cfu/g) of bacteria of the Bacillus genus. In a further example, the composition described herein comprises about 12,000 cfu/g (i.e. about 1.2×10⁴ cfu/g) of bacteria of the Bacillus genus.

In another example, the composition described herein comprises about 13,000 cfu/g (i.e. about 1.3×10⁴ cfu/g) of bacteria of the Bacillus genus.

In another example, the composition described herein comprises about 14,000 cfu/g (i.e. about 1.4×10⁴ cfu/g) of bacteria of the Bacillus genus. In a further example, the composition described herein comprises about 15,000 cfu/g (i.e. about 1.5×10⁴ cfu/g) of bacteria of the Bacillus genus.

A person of skill in the art would readily be able to determine the amount or concentration of bacteria present in a composition using routine methods known in the art. For instance, the total viable count (TVC), of Bacillus live cells for example can be determined by established cultivation methods based on specific Bacillus media, such Chrome Select agar. Alternative methods are known in the art.

In one example, the composition described herein comprises about 5,000 cfu of bacteria of the Bacillus genus/dose to about 1×10⁸ cfu of bacteria of the Bacillus genus/dose.

In one example, the composition described herein comprises at least about 1×10⁸ cfu of bacteria of the Bacillus genus/dose. In another example, the composition described herein comprises at least about 1×10⁷ cfu of bacteria of the Bacillus genus/dose. In another example, the composition described herein comprises at least about 1×10⁶ cfu of bacteria of the Bacillus genus/dose.

In a further example, the composition described herein comprises at least about 1×10⁵ cfu of bacteria of the Bacillus genus/dose.

In one example, the composition described herein comprises at least about 5,000 cfu (i.e. at least about 0.5×10⁴ cfu) of bacteria of the Bacillus genus/dose.

In another example, the composition described herein comprises at least about 10,000 cfu (i.e. at least about 1.0×10⁴ cfu) of bacteria of the Bacillus genus/dose. In another example, the composition described herein comprises at least about 11,000 cfu (i.e. at least about 1.1×10⁴ cfu) of bacteria of the Bacillus genus/dose. In a further example, the composition described herein comprises at least about 12,000 cfu (i.e. at least about 1.2×10⁴ cfu) of bacteria of the Bacillus genus/dose. In another example, the composition described herein comprises at least about 13,000 cfu (i.e. at least about 1.3×10⁴ cfu) of bacteria of the Bacillus genus/dose. In another example, the composition described herein comprises at least about 14,000 cfu (i.e. at least about 1.4×10⁴ cfu) of bacteria of the Bacillus genus/dose. In another example, the composition described herein comprises at least about 15,000 cfu (i.e. at least about 1.5×10⁴ cfu) of bacteria of the Bacillus genus/dose.

In one example, the composition described herein comprises about 1×10⁸ cfu of bacteria of the Bacillus genus/dose. In another example, the composition described herein comprises about 1×10⁷ cfu of bacteria of the Bacillus genus/dose. In another example, the composition described herein comprises about 1×10⁶ cfu of bacteria of the Bacillus genus/dose.

In a further example, the composition described herein comprises about 1×10⁵ cfu of bacteria of the Bacillus genus/dose.

In one example, the composition described herein comprises about 5,000 cfu (i.e. about 0.5×10⁴ cfu) of bacteria of the Bacillus genus/dose. In one example, the composition described herein comprises about 10,000 cfu (i.e. about 1.0×10⁴ cfu) of bacteria of the Bacillus genus/dose. In another example, the composition described herein comprises about 11,000 cfu (i.e. about 1.1×10⁴ cfu) of bacteria of the Bacillus genus/dose. In a further example, the composition described herein comprises about 12,000 cfu (i.e. about 1.2×10⁴ cfu) of bacteria of the Bacillus genus/dose. In another example, the composition described herein comprises about 13,000 cfu (i.e. about 1.3×10⁴ cfu) of bacteria of the Bacillus genus/dose. In another example, the composition described herein comprises about 14,000 cfu (i.e. about 1.4×10⁴ cfu) of bacteria of the Bacillus genus/dose. In a further example, the composition described herein comprises about 15,000 cfu (i.e. about 1.5×10⁴ cfu) of bacteria of the Bacillus genus/dose.

As would be clear to the skilled person, the amount or concentration of the one or more bacterial species of the Bacillus genus in the composition may be made up from any individual species of the Bacillus genus or any combination of species of the Bacillus genus. For example, the amount or concentration of the one or more bacterial species of the Bacillus genus in the composition may be made up from entirely one Bacillus species (e.g. the concentration may be made up from entirely B. subtilis or entirely B. coagulans). Alternatively, the amount or concentration of the one or more bacterial species of the Bacillus genus in the composition may be made up from two or more, three or more, four or more, or five or more bacterial species of the Bacillus genus (e.g. the concentration may be made up from B. subtilis and B. coagulans). Accordingly, in one example, the amount or concentration of the one or more bacterial species of the Bacillus genus in the composition refers to the amount or concentration of the combination of the Bacillus species present.

In some examples, the composition provided herein comprises an appropriate concentration of bacteria, wherein a proportion of the concentration is made up from one or more bacterial species of the Bacillus genus (e.g. B. subtilis and/or B. coagulans). In one example, the composition described herein comprises about 10,000 cfu/g (i.e. about 1.0×10⁴ cfu/g) of bacteria to about 1×10⁸ cfu/g of bacteria. In one example, the composition described herein comprises at least about 1×10⁸ cfu/g of bacteria. In another example, the composition described herein comprises at least about 1×10⁷ cfu/g of bacteria. In another example, the composition described herein comprises at least about 1×10⁶ cfu/g of bacteria. In a further example, the composition described herein comprises at least about 1×10⁵ cfu/g of bacteria. In one example, the composition described herein comprises at least about 10,000 cfu/g (i.e. at least about 1.0×10⁴ cfu/g) of bacteria. In another example, the composition described herein comprises at least about 11,000 cfu/g (i.e. at least about 1.1×10⁴ cfu/g) of bacteria. In a further example, the composition described herein comprises at least about 12,000 cfu/g (i.e. at least about 1.2×10⁴ cfu/g) of bacteria. In another example, the composition described herein comprises at least about 13,000 cfu/g (i.e. at least about 1.3×10⁴ cfu/g) of bacteria. In another example, the composition described herein comprises at least about 14,000 cfu/g (i.e. at least about 1.4×10⁴ cfu/g) of bacteria. In another example, the composition described herein comprises at least about 15,000 cfu/g (i.e. at least about 1.5×10⁴ cfu/g) of bacteria. In one example, the composition described herein comprises about 1×10⁸ cfu/g of bacteria. In another example, the composition described herein comprises about 1×10⁷ cfu/g of bacteria. In another example, the composition described herein comprises about 1×10⁶ cfu/g of bacteria. In a further example, the composition described herein comprises about 1×10⁵ cfu/g of bacteria. In one example, the composition described herein comprises about 10,000 cfu/g (i.e. about 1.0×10⁴ cfu/g) of bacteria. In another example, the composition described herein comprises about 11,000 cfu/g (i.e. about 1.1×10⁴ cfu/g) of bacteria. In a further example, the composition described herein comprises about 12,000 cfu/g (i.e. about 1.2×10⁴ cfu/g) of bacteria. In another example, the composition described herein comprises about 13,000 cfu/g (i.e. about 1.3×10⁴ cfu/g) of bacteria. In another example, the composition described herein comprises about 14,000 cfu/g (i.e. about 1.4×10⁴ cfu/g) of bacteria. In a further example, the composition described herein comprises about 15,000 cfu/g (i.e. about 1.5×10⁴ cfu/g) of bacteria.

In some examples, the composition provided herein comprises an appropriate amount of bacteria, wherein a proportion of the amount is made up from one or more bacterial species of the Bacillus genus (e.g. B. subtilis and/or B. coagulans).

In one example, the composition described herein comprises about 5,000 cfu of bacteria/dose to about 1×10⁸ cfu of bacteria/dose. In one example, the composition described herein comprises at least about 1×10⁸ cfu of bacteria/dose. In another example, the composition described herein comprises at least about 1×10⁷ cfu of bacteria/dose. In another example, the composition described herein comprises at least about 1×10⁶ cfu of bacteria/dose. In a further example, the composition described herein comprises at least about 1×10⁵ cfu of bacteria/dose. In one example, the composition described herein comprises at least about 5,000 cfu (i.e. at least about 0.5×10⁴ cfu) of bacteria/dose. In another example, the composition described herein comprises at least about 10,000 cfu (i.e. at least about 1.0×10⁴ cfu) of bacteria/dose. In another example, the composition described herein comprises at least about 11,000 cfu (i.e. at least about 1.1×10⁴ cfu) of bacteria/dose. In a further example, the composition described herein comprises at least about 12,000 cfu (i.e. at least about 1.2×10⁴ cfu) of bacteria/dose. In another example, the composition described herein comprises at least about 13,000 cfu (i.e. at least about 1.3×10⁴ cfu) of bacteria/dose. In another example, the composition described herein comprises at least about 14,000 cfu (i.e. at least about 1.4×10⁴ cfu) of bacteria/dose. In another example, the composition described herein comprises at least about 15,000 cfu (i.e. at least about 1.5×10⁴ cfu) of bacteria/dose. In one example, the composition described herein comprises about 1×10⁸ cfu of bacteria/dose. In another example, the composition described herein comprises about 1×10⁷ cfu of bacteria/dose. In another example, the composition described herein comprises about 1×10⁶ cfu of bacteria/dose. In a further example, the composition described herein comprises about 1×10¹ cfu of bacteria/dose. In one example, the composition described herein comprises about 5,000 cfu (i.e. about 0.5×10⁴ cfu) of bacteria/dose. In one example, the composition described herein comprises about 10,000 cfu (i.e. about 1.0×10⁴ cfu) of bacteria/dose. In another example, the composition described herein comprises about 11,000 cfu (i.e. about 1.1×10⁴ cfu) of bacteria/dose. In a further example, the composition described herein comprises about 12,000 cfu (i.e. about 1.2×10⁴ cfu) of bacteria/dose. In another example, the composition described herein comprises about 13,000 cfu (i.e. about 1.3×10⁴ cfu) of bacteria/dose. In another example, the composition described herein comprises about 14,000 cfu (i.e. about 1.4×10⁴ cfu) of bacteria/dose. In a further example, the composition described herein comprises about 15,000 cfu (i.e. about 1.5×10⁴ cfu) of bacteria/dose.

As would be clear to the skilled person, the amount or concentration of bacteria (wherein a proportion is made up from one or more bacterial species of the Bacillus genus (e.g. B. subtilis and/or B. coagulans) may further comprise any appropriate individual species or any combination of species. For example, the amount or concentration of bacteria (wherein a proportion is made up from one or more bacterial species of the Bacillus genus (e.g. B. subtilis and/or B. coagulans)) may further comprise a proportion of one or more bacterial species selected from the group consisting of: Bacillus amyloliquefaciens, Bacillus velezensis, Bacillus sp MT 03, Bacillus atrophaeus, and Pediococcus pentosaceus.

In one example, the one or more bacterial species of the Bacillus genus is genetically modified.

As used herein, “genetic modification” and “genetic engineering” refer to the direct manipulation (e.g. modification) of one or more genes, for example, using recombinant DNA technology. Traditionally, humans have manipulated genomes indirectly by controlling breeding and selecting offspring with desired traits however genetic engineering involves direct manipulation (e.g. modification) of one or more genes. For example, a gene from another species may be added to an organism's genome to give it a desired phenotype.

In other examples, the one or more bacterial species of the Bacillus genus is not genetically modified. In other words, the one or more bacterial species of the Bacillus genus may be naturally occurring.

The one or more bacterial species of the Bacillus genus may be present within a microbial consortium. “Microbial consortium”, as used herein, refers to a group of microbes (e.g. bacteria) wherein the group comprises two or more distinct microbes (e.g. two of more bacteria which may be from the same species (e.g. two or more distinct strains) or distinct species (e.g. two or more distinct species).

The microbial consortium may be a naturally occurring microbial consortium (e.g. a consortium that is naturally produced during fermentation of a cereal, such as rice bran). In other words, the one or more bacterial species of the Bacillus genus may be part of the composition by virtue of the presence of a fermented cereal, such as rice bran (with its associated naturally produced microbial consortium) in the composition. The one or more bacterial species of the Bacillus genus may therefore be a natural component of a fermented cereal, such as rice bran.

The inventors have found that Bacillus subtilis strain DFM 0326 (LMG P-32899), Bacillus subtilis strain DFM 1015 (LMG P-32900) and Bacillus coagulans strain DFM 0705 (LMG P-32921) can be isolated from the compositions (in particular, the fermented rice bran) used in the examples provided herein. Accordingly, the B. subtilis strain DFM 0326 (LMG P-32899) and Bacillus subtilis strain DFM 1015 (LMG P-32900), and the B. coagulans strain DFM 0705 (LMG P-32921) may be part of the composition described herein by virtue of the presence of a fermented cereal, such as rice bran (with its associated naturally produced microbial consortium), in the composition.

In another example, the one or more bacterial species selected from the group consisting of: Bacillus amyloliquefaciens, Bacillus velezensis, Bacillus sp MT 03, Bacillus atrophaeus, and Pediococcus pentosaceus may be (part of) a naturally occurring microbial consortium (e.g. a consortium that is naturally produced during fermentation of a cereal, such as rice bran). In other words, the one or more bacterial species selected from the group consisting of: Bacillus amyloliquefaciens, Bacillus velezensis, Bacillus sp MT 03, Bacillus atrophaeus, and Pediococcus pentosaceus may be part of the composition by virtue of the presence of a fermented cereal, such as rice bran (with its associated naturally produced microbial consortium) in the composition. The one or more bacterial species selected from the group consisting of: Bacillus amyloliquefaciens, Bacillus velezensis, Bacillus sp MT 03, Bacillus atrophaeus, and Pediococcus pentosaceus may therefore be a natural component of a fermented cereal, such as rice bran.

In another example, the one or more bacterial species of the Bacillus genus (e.g. B.subtilis and/or B. coagulans) and the one or more bacterial species selected from the group consisting of: Bacillus amyloliquefaciens, Bacillus velezensis, Bacillus sp MT 03, Bacillus atrophaeus, and Pediococcus pentosaceus may be (part of) a naturally occurring microbial consortium (e.g. a consortium that is naturally produced during fermentation of a cereal, such as rice bran). In other words, the one or more bacterial species of the Bacillus genus (e.g. B. subtilis and/or B. coagulans) and the one or more bacterial species selected from the group consisting of: Bacillus amyloliquefaciens, Bacillus velezensis, Bacillus sp MT 03, Bacillus atrophaeus, and Pediococcus pentosaceus may be part of the composition by virtue of the presence of a fermented cereal, such as rice bran (with its associated naturally produced microbial consortium) in the composition. The one or more bacterial species of the Bacillus genus (e.g. B. subtilis and/or B. coagulans) and the one or more bacterial species selected from the group consisting of: Bacillus amyloliquefaciens, Bacillus velezensis, Bacillus sp MT 03, Bacillus atrophaeus, and Pediococcus pentosaceus may therefore be a natural component of a fermented cereal, such as rice bran.

As would be clear to a person of skill in the art, the invention is not limited to naturally occurring microbial consortia. Accordingly, the microbial consortia discussed above may also be generated artificially, for example by combining one or more bacterial isolates together. In one example, the one or more bacterial species of the Bacillus genus (e.g. B. subtilis and/or B. coagulans) may therefore be added to the composition individually.

The alcohol degrading composition may therefore also include a cereal such as rice bran (e.g. fermented rice bran) as discussed in more detail elsewhere herein. The fermented rice bran, for example, may be the natural source of the one or more bacterial species of the Bacillus genus (e.g. B. subtilis and/or B. coagulans) present within the composition.

The inventors have surprisingly discovered that L-cysteine can be used to increase alcohol degradation by one or more bacterial species of the Bacillus genus particularly when combined with a high molecular weight low osmolality carbohydrate, such as dextrin.

The alcohol degrading composition described herein thus comprises L-cysteine in combination with the one or more bacterial species of the Bacillus genus (e.g. wherein the bacteria are added to the composition as a bacterial supplement or as a natural product e.g. as part of fermented rice bran).

L-cysteine (L-cys) is a non-essential amino acid and thus is one of the building blocks required for the synthesis of proteins. It contains sulphur in the form of a thiol group (—SH) at the end of its side chain. The —SH group is responsible for the high reactive capacity of the amino acid, and therefore is responsible for many of its biological functions in human beings. L-cysteine occupies a key position in sulfur metabolism in all organisms and is used in the synthesis of proteins, glutathione, biotin, lipoic acid, methionine and other sulfur-containing metabolites. In addition, L-cysteine serves as a precursor for the biosynthesis of coenzyme A. The biosynthesis of L-cysteine has been studied in detail in bacteria, especially in enterobacteria. The amino acid L-cysteine is not only of biological importance but is also of economic importance. It is used, for example, as a food additive (in particular in the baking industry), as a starting material in cosmetics, and as a starting material for the preparation of active pharmaceutical ingredients (in particular N-acetyl-cysteine and S-carboxymethyl-cysteine).

As would be clear to a person of skill in the art, reference to L-cysteine herein refers to the amino acid L-cysteine in any suitable form. The term “L-cysteine” therefore encompasses L-cysteine in free form as well as L-cysteine salts.

In the context of the present invention, in one example L-cysteine can be in the free form, a salt thereof, or a mixture thereof.

Examples of the salt include, for example, sulfate, hydrochloride, carbonate, ammonium salt, sodium salt, and potassium salt. In one example, the L-cysteine is a crystal fraction of >0.1 mm.

L-cysteine is available from several suppliers and can be readily sourced by the skilled person. Furthermore, the skilled person would readily be able to detect the presence of L-cysteine in a substance (e.g. in a composition described herein) using methods known in the art. For instance, L-Cysteine crystals may be macroscopically detected in the composition described herein as white particles. HPLC, High-Performance Liquid Chromatography, on an Inertsil ODs-3 column is an established method for detection of L-cysteine.

L-cysteine may be obtained industrially by hydrolysis of animal materials, such as poultry feathers or hog hair. In contrast, synthetic L-cys may be obtained from fermentation of genetically modified E. coli or Pseudomonas thiazolinophilum. Accordingly, in some examples, L-cysteine is of animal origin. In other examples, L-cysteine is of synthetic origin.

In one example, L-cysteine is of vegetable origin.

In some examples, the composition described herein may comprise an L-cysteine derivative instead of or in addition to L-cysteine.

L-cysteine derivatives are well known to persons of skill in the art. N-acetyl cysteine (NAC) is an N-acetylated form of the amino acid L-cysteine thus is an example of an L-cysteine derivative. Accordingly, in some examples the composition described herein may comprise N-acetylcysteine (NAC). NAC is readily available in the art.

L-cysteine is present in the alcohol degrading composition described herein at an appropriate concentration or amount. The amount or concentration of L-cysteine may therefore be described by reference to the % w/w of composition or by the total weight (e.g. mgs) per dose of composition (in other words the weight per effective dose). As would be clear to a person of skill in the art, and as described in more detail elsewhere herein, a dose may include one or more dosage units (e.g. 2 dosage units). In examples wherein a plurality of dosage units are used to provide an effective dose, the weight per dose corresponds to the total weight of L-cysteine over the plurality of dosage units.

In one example, the composition comprises about 10% w/w of L-cysteine to about 40% w/w of L-cysteine.

In another example, the composition comprises about 15% w/w of L-cysteine to about 35% w/w of L-cysteine. In another example, the composition comprises about 20% w/w of L-cysteine to about 30% w/w of L-cysteine.

In one example, the composition comprises at least about 10% w/w of L-cysteine.

In another example, the composition comprises at least about 15% w/w of L-cysteine. In a further example, the composition comprises at least about 20% w/w of L-cysteine. In another example, the composition comprises at least about 25% w/w of L-cysteine. In another example, the composition comprises at least about 30% w/w of L-cysteine. In a further example, the composition comprises at least about 35% w/w of L-cysteine. In a further example, the composition comprises at least about 40% w/w of L-cysteine.

In some examples, the composition comprises about 10% w/w of L-cysteine.

In another example, the composition comprises about 15% w/w of L-cysteine. In another example, the composition comprises about 20% w/w of L-cysteine. In a further example, the composition comprises about 25% w/w of L-cysteine. In another example, the composition comprises about 30% w/w of L-cysteine. In another example, the composition comprises about 35% w/w of L-cysteine. In a further example, the composition comprises about 40% w/w of L-cysteine.

In one example, the composition comprises about 38 mg of L-cysteine/dose to about 200 mg of L-cysteine/dose.

In one example, the composition comprises at least about 38 mg of L-cysteine/dose.

In one example, the composition comprises at least about 50 mg of L-cysteine/dose. In one example, the composition comprises at least about 75 mg of L-cysteine/dose. In one example, the composition comprises at least about 100 mg of L-cysteine/dose.

In one example, the composition comprises at least about 150 mg of L-cysteine/dose.

In one example, the composition comprises at least about 160 mg of L-cysteine/dose.

In one example, the composition comprises at least about 180 mg of L-cysteine/dose.

In one example, the composition comprises about 38 mg of L-cysteine/dose.

In one example, the composition comprises about 50 mg of L-cysteine/dose. In one example, the composition comprises about 75 mg of L-cysteine/dose. In one example, the composition comprises about 100 mg of L-cysteine/dose.

In one example, the composition comprises about 150 mg of L-cysteine/dose.

In one example, the composition comprises about 160 mg of L-cysteine/dose. In one example, the composition comprises about 180 mg of L-cysteine/dose.

The data presented herein shows that the addition of L-cysteine to a composition comprising a high molecular weight low osmolality carbohydrate (e.g. dextrin) and one or more bacterial species of the Bacillus genus results in re-programing of the bacteria so that they more efficiently degrade alcohol compared to the rate of alcohol degradation in the absence of L-cysteine.

The alcohol degrading composition described herein therefore comprises L-cysteine in combination with the one or more bacterial species of the Bacillus genus and a high molecular weight low osmolality carbohydrate (e.g. dextrin).

As used herein, a “high molecular weight low osmolality carbohydrate” refers to a carbohydrate with a molecular weight of about 500,000 g/mol to about 700,000 g/mol wherein the osmolality of the carbohydrate is low. A person of skill in the art would readily be able to identify appropriate carbohydrates with a low osmolality using routine tests known in the art. For the avoidance of doubt, as used herein, a low osmolality carbohydrate is a carbohydrate that has about 50% greater glycogen recovery than maltodextrin.

The high molecular weight low osmolality carbohydrate is present in the alcohol degrading composition described herein at an appropriate concentration or amount. The amount or concentration of the high molecular weight low osmolality carbohydrate may therefore be described by reference to the % w/w of composition or by the total weight (e.g. mgs) per dose of composition (in other words the weight per effective dose). As would be clear to a person of skill in the art, and as described in more detail elsewhere herein, a dose may include one or more dosage units (e.g. 2 dosage units). In examples wherein a plurality of dosage units are used to provide an effective dose, the weight per dose corresponds to the total weight of the high molecular weight low osmolality carbohydrate over the plurality of dosage units.

In one example, the composition comprises about 0.5% w/w to about 5% w/w of high molecular weight low osmolality carbohydrate.

In another example, the composition comprises about 0.5% w/w to about 3% w/w of high molecular weight low osmolality carbohydrate. In one example, the composition comprises about 0.5% w/w to about 2% w/w of high molecular weight low osmolality carbohydrate.

In one example, the composition comprises at least about 0.5% w/w of high molecular weight low osmolality carbohydrate.

In another example, the composition comprises at least about 1% w/w of high molecular weight low osmolality carbohydrate. In another example, the composition comprises at least about 1.5% w/w of high molecular weight low osmolality carbohydrate. In a further example, the composition comprises at least about 2% w/w of high molecular weight low osmolality carbohydrate. In another example, the composition comprises at least about 2.5% w/w of high molecular weight low osmolality carbohydrate. In another example, the composition comprises at least about 3% w/w of high molecular weight low osmolality carbohydrate. In another example, the composition comprises at least about 3.5% w/w of high molecular weight low osmolality carbohydrate. In a further example, the composition comprises at least about 4% w/w of high molecular weight low osmolality carbohydrate. In another example, the composition comprises at least about 4.5% w/w of high molecular weight low osmolality carbohydrate. In a further example, the composition comprises at least about 5% w/w of high molecular weight low osmolality carbohydrate.

In one example, the composition comprises about 0.5% w/w of high molecular weight low osmolality carbohydrate.

In another example, the composition comprises about 1% w/w of high molecular weight low osmolality carbohydrate. In another example, the composition comprises about 1.5% w/w of high molecular weight low osmolality carbohydrate. In a further example, the composition comprises about 2% w/w of high molecular weight low osmolality carbohydrate. In another example, the composition comprises about 2.5% w/w of high molecular weight low osmolality carbohydrate. In another example, the composition comprises about 3% w/w of high molecular weight low osmolality carbohydrate. In another example, the composition comprises about 3.5% w/w of high molecular weight low osmolality carbohydrate. In a further example, the composition comprises about 4% w/w of high molecular weight low osmolality carbohydrate. In another example, the composition comprises about 4.5% w/w of high molecular weight low osmolality carbohydrate. In a further example, the composition comprises about 5% w/w of high molecular weight low osmolality carbohydrate.

In one example, the composition comprises about 2 mg to about 50 mg of high molecular weight low osmolality carbohydrate/dose.

In one example, the composition comprises at least about 2 mg of high molecular weight low osmolality carbohydrate/dose.

In one example, the composition comprises at least about 2 mg of high molecular weight low osmolality carbohydrate/dose.

In one example, the composition comprises at least about 4 mg of high molecular weight low osmolality carbohydrate/dose.

In one example, the composition comprises at least about 10 mg of high molecular weight low osmolality carbohydrate/dose. In one example, the composition comprises at least about 15 mg of high molecular weight low osmolality carbohydrate/dose. In one example, the composition comprises at least about 20 mg of high molecular weight low osmolality carbohydrate/dose. In one example, the composition comprises at least about 25 mg of high molecular weight low osmolality carbohydrate/dose. In one example, the composition comprises at least about 30 mg of high molecular weight low osmolality carbohydrate/dose. In one example, the composition comprises at least about 35 mg of high molecular weight low osmolality carbohydrate/dose. In one example, the composition comprises at least about 40 mg of high molecular weight low osmolality carbohydrate/dose. In one example, the composition comprises at least about 45 mg of high molecular weight low osmolality carbohydrate/dose.

In one example, the composition comprises about 2 mg of high molecular weight low osmolality carbohydrate/dose.

In one example, the composition comprises about 4 mg of high molecular weight low osmolality carbohydrate/dose.

In one example, the composition comprises about 10 mg of high molecular weight low osmolality carbohydrate/dose. In one example, the composition comprises about 15 mg of high molecular weight low osmolality carbohydrate/dose. In one example, the composition comprises about 20 mg of high molecular weight low osmolality carbohydrate/dose. In one example, the composition comprises about 25 mg of high molecular weight low osmolality carbohydrate/dose. In one example, the composition comprises about 30 mg of high molecular weight low osmolality carbohydrate/dose. In one example, the composition comprises about 35 mg of high molecular weight low osmolality carbohydrate/dose. In one example, the composition comprises about 40 mg of high molecular weight low osmolality carbohydrate/dose. In one example, the composition comprises about 45 mg of high molecular weight low osmolality carbohydrate/dose.

In one example, the high molecular weight low osmolality carbohydrate is dextrin.

Dextrin is a generic term applied to a variety of products obtained by heating a starch in the presence of small amounts of moisture and an acid. Dextrins are a group of low-molecular-weight carbohydrates produced by the hydrolysis of starch or glycogen. “Dextrin” refers to a glucose polymer produced by the hydrolysis of starch (or glycogen) which comprises glucose units linked together by means mainly of α-1,4 linkages. In addition to α-1,4 linkages, there may be a proportion of α-1,6 linkages in a particular dextrin, the amount depending on the starch starting material. Since the rate of biodegradability of α-1,6 linkages is typically less than that for α-1,4 linkages, it is preferred that, for many applications, the percentage of α-1,6 linkages is less than 10% and more preferably less than 5%. In some examples, dextrins are thus mixtures of polymers of D-glucose units linked by alpha-(1->4) or alpha-(1->6) glycosidic bonds.

Dextrins can be produced from starch using enzymes like amylases, as during digestion in the human body and during malting and mashing, or by applying dry heat under acidic conditions (pyrolysis or roasting). The latter process is used industrially. Dextrins produced by heat are also known as pyrodextrins. Typically, dextrins are produced by the hydrolysis of starch obtained from various natural products such as wheat, rice, corn, maize and tapioca.

Dextrins are typically white, yellow, or brown powder that are partially or fully water-soluble, yielding optically active solutions of low viscosity. Dextrins are available from several suppliers and can be readily sourced by the skilled person. The skilled person would readily be able to identify suitable dextrins for use in the context of the present invention. The skilled person would readily be able to detect the presence of dextrin in a substance (e.g. in a composition described herein) using methods known in the art. For example, most dextrins can be detected with iodine solution.

The term “dextrin” encompasses pyrodextrins, digestible dextrins, and hydrogenated products thereof, including derivatives thereof. The term “dextrin derivative” herein means those obtained by chemically or enzymatically processing dextrins, and encompasses, for example, branched dextrins obtained by causing a glycosyltransferase to act on a dextrin, and cyclodextrins obtained by causing a cyclodextrin producing enzyme to act on a starch, in addition to the above-described polydextrose.

In some examples, dextrin is enzymatically processed.

In one example, the dextrin, and thus, the high molecular weight low osmolality carbohydrate, is cluster dextrin (also referred to as highly branched cyclic dextrin). Cluster dextrin is a maltodextrin which has a high molecular weight, but narrow weight distribution, is soluble, and its osmotic pressure is near zero. Typical macromolecular carbohydrates are less soluble than Cluster Dextrin. On the other hand low molecular carbohydrates exhibit higher osmotic pressure, as does pure glucose solution. Along with other digestible contents typically found in sports drinks, this slows their descent to the small intestine. Cluster Dextrin is faster to the small intestine and faster to ramp-up endurance. Solubility is at the heart of Cluster Dextrin's functioning. High molecular weight Cluster Dextrin also degrades slowly, balancing insulin secretion with lipid breakdown. Cluster dextrin is manufactured by Glico Nutrition.

In some examples, the dextrin, and thus, the high molecular weight low osmolality carbohydrate is derived from wheat or corn (e.g. in some examples the dextrin is wheat dextrin or corn dextrin).

In some examples, the dextrin is enzymatically processed wheat or corn.

In one example, the dextrin is derived from wheat (e.g. wheat dextrin). For instance, the dextrin may be a wheat dextrin powder (such as Surbex Nutri-Fiber Wheat Dextrin Powder which is a soluble non viscous fiber).

In another example, the dextrin is derived from corn (e.g. corn dextrin).

In a particular example, the dextrin is hydrolysed corn dextrin (e.g. Vitargo).

In some examples, dextrin is present in the alcohol degrading composition described herein at an appropriate concentration or amount.

In one example, the composition comprises about 0.5% w/w to about 5% w/w of dextrin.

In another example, the composition comprises about 0.5% w/w to about 3% w/w of dextrin. In another example, the composition comprises about 0.5% w/w to about 2% w/w of dextrin.

In one example, the composition comprises at least about 0.5% w/w of dextrin.

In another example, the composition comprises at least about 1% w/w of dextrin. In another example, the composition comprises at least about 1.5% w/w of dextrin. In a further example, the composition comprises at least about 2% w/w of dextrin. In another example, the composition comprises at least about 2.5% w/w of dextrin. In another example, the composition comprises at least about 3% w/w of dextrin. In another example, the composition comprises at least about 3.5% w/w of dextrin. In a further example, the composition comprises at least about 4% w/w of dextrin. In another example, the composition comprises at least about 4.5% w/w of dextrin. In a further example, the composition comprises at least about 5% w/w of dextrin.

In one example, the composition comprises about 0.5% w/w of dextrin.

In another example, the composition comprises about 1% w/w of dextrin. In another example, the composition comprises about 1.5% w/w of dextrin. In a further example, the composition comprises about 2% w/w of dextrin. In another example, the composition comprises about 2.5% w/w of dextrin. In another example, the composition comprises about 3% w/w of dextrin. In another example, the composition comprises about 3.5% w/w of dextrin. In a further example, the composition comprises about 4% w/w of dextrin. In another example, the composition comprises about 4.5% w/w of dextrin. In a further example, the composition comprises about 5% w/w of dextrin.

In one example, the composition comprises about 2 mg to about 50 mg of dextrin/dose.

In one example, the composition comprises at least about 2 mg of dextrin/dose.

In one example, the composition comprises at least about 4 mg of dextrin/dose.

In one example, the composition comprises at least about 5 mg of dextrin/dose. In one example, the composition comprises at least about 10 mg of dextrin/dose. In one example, the composition comprises at least about 15 mg of dextrin/dose. In one example, the composition comprises at least about 20 mg of dextrin/dose. In one example, the composition comprises at least about 25 mg of dextrin/dose. In one example, the composition comprises at least about 30 mg of dextrin/dose. In one example, the composition comprises at least about 35 mg of dextrin/dose. In one example, the composition comprises at least about 40 mg of dextrin/dose. In one example, the composition comprises at least about 45 mg of dextrin/dose.

In one example, the composition comprises about 2 mg of dextrin/dose.

In one example, the composition comprises about 4 mg of dextrin/dose.

In one example, the composition comprises about 10 mg of dextrin/dose. In one example, the composition comprises about 15 mg of dextrin/dose. In one example, the composition comprises about 20 mg of dextrin/dose. In one example, the composition comprises about 25 mg of dextrin/dose. In one example, the composition comprises about 30 mg of dextrin/dose. In one example, the composition comprises about 35 mg of dextrin/dose. In one example, the composition comprises about 40 mg of dextrin/dose. In one example, the composition comprises about 45 mg of dextrin/dose.

In one example, the composition described herein comprises L-cysteine and dextrin. L-cysteine and dextrin may be present in the alcohol degrading composition described herein at any appropriate concentration. Appropriate concentrations or amounts of L-cysteine and dextrin are described elsewhere herein and apply equally to a composition described herein comprising both L-cysteine and dextrin. Illustrative examples of appropriate concentrations or amounts are provided below.

In one example, the composition comprises about 0.5% w/w to 5% w/w of dextrin and about 10% w/w of L-cysteine to about 40% w/w of L-cysteine. In each of these examples, the composition may also comprise about 50% w/w to about 90% w/w of rice bran (e.g. fermented rice bran) and/or about 10,000 cfu/g of bacteria of the Bacillus genus to about 15,000 cfu/g of bacteria of the Bacillus genus (e.g. B. subtilis and/or B. coagulans).

In one example, the composition described herein comprises at least about 0.5% w/w of dextrin and at least about 10% w/w of L-cysteine. For example, the composition described herein may comprise about 0.5% w/w of dextrin and about 10% w/w of L-cysteine. This example corresponds with a composition as tested in example 1, test 7B. In each of these examples, the composition may also comprise at least about 67% w/w of rice bran (e.g. at least about 67% w/w of fermented rice bran) and/or at least 10,000 cfu/g of bacteria of the Bacillus genus (e.g. B. subtilis and/or B. coagulans). For instance, in each of these examples, the composition may comprise about 67% w/w of rice bran (e.g. about 67% w/w of fermented rice bran) and/or 10,000 cfu/g of bacteria of the Bacillus genus (e.g. B. subtilis and/or B. coagulans).

In one example, the composition described herein comprises at least about 0.5% w/w of dextrin and at least about 20% w/w of L-cysteine. For example, the composition described herein may comprise about 0.5% w/w of dextrin and about 20% w/w of L-cysteine. This example corresponds with a composition as tested in example 1, test 6B. In each of these examples, the composition may also comprise at least about 67% w/w of rice bran (e.g. at least about 67% w/w of fermented rice bran) and/or at least 10,000 cfu/g of bacteria of the Bacillus genus (e.g. B. subtilis and/or B. coagulans). For instance, in each of these examples, the composition may comprise about 67% w/w of rice bran (e.g. about 67% w/w of fermented rice bran) and/or 10,000 cfu/g of bacteria of the Bacillus genus (e.g. B. subtilis and/or B. coagulans).

In one example, the composition described herein comprises at least about 0.5% w/w of dextrin and at least about 30% w/w of L-cysteine. For example, the composition described herein may comprise about 0.5% w/w of dextrin and about 30% w/w of L-cysteine. This example corresponds with a composition as tested in example 1, test 5B. In each of these examples, the composition may also comprise at least about 67% w/w of rice bran (e.g. at least about 67% w/w of fermented rice bran) and/or at least 10,000 cfu/g of bacteria of the Bacillus genus (e.g. B. subtilis and/or B. coagulans). For instance, in each of these examples, the composition may comprise about 67% w/w of rice bran (e.g. about 67% w/w of fermented rice bran) and/or 10,000 cfu/g of bacteria of the Bacillus genus (e.g. B. subtilis and/or B. coagulans).

In one example, the composition described herein comprises at least about 5% w/w of dextrin and at least about 20% w/w of L-cysteine. For example, the composition described herein may comprise about 5% w/w of dextrin and about 20% w/w of L-cysteine. This example corresponds with a composition as tested in example 1, test 8B. In each of these examples, the composition may also comprise at least about 67% w/w of rice bran (e.g. at least about 67% w/w of fermented rice bran) and/or at least 10,000 cfu/g of bacteria of the Bacillus genus (e.g. B. subtilis and/or B. coagulans). For instance, in each of these examples, the composition may comprise about 67% w/w of rice bran (e.g. about 67% w/w of fermented rice bran) and/or 10,000 cfu/g of bacteria of the Bacillus genus (e.g. B. subtilis and/or B. coagulans).

In one example, the composition described herein comprises at least about 3% w/w of dextrin and at least about 30% w/w of L-cysteine. For example, the composition described herein may comprise about 3% w/w of dextrin and about 30% w/w of L-cysteine. In each of these examples, the composition may also comprise at least about 67% w/w of rice bran (e.g. at least about 67% w/w of fermented rice bran) and/or at least 10,000 cfu/g of bacteria of the Bacillus genus (e.g. B. subtilis and/or B. coagulans). For instance, in each of these examples, the composition may comprise about 67% w/w of rice bran (e.g. about 67% w/w of fermented rice bran) and/or 10,000 cfu/g of bacteria of the Bacillus genus (e.g. B. subtilis and/or B. coagulans).

In one example, the composition comprises about 2 mg to about 50 mg of dextrin/dose and about 38 mg to about 200 mg of L-cysteine/dose. In each of these examples, the composition may also comprise about 300 mg to about 600 mg of rice bran/dose (e.g. fermented rice bran/dose) and/or about 5,000 cfu of bacteria of the Bacillus genus/dose to about 1×10⁸ cfu of bacteria of the Bacillus genus/dose (e.g. B. subtilis and/or B. coagulans).

In one example, the composition described herein comprises at least about 2 mg of dextrin/dose and at least about 38 mg of L-cysteine/dose. For example, the composition described herein may comprise about 2 mg of dextrin/dose and about 38 mg of L-cysteine/dose. This example corresponds with a composition as tested in example 1, test 7B. In each of these examples, the composition may also comprise at least about 300 mg of rice bran/dose (e.g. at least about 300 mg of fermented rice bran/dose) and/or at least 5,000 cfu of bacteria of the Bacillus genus/dose (e.g. B. subtilis and/or B. coagulans). For instance, in each of these examples, the composition may comprise about 300 mg of rice bran/dose (e.g. about 300 mg of fermented rice bran/dose) and/or 5,000 cfu of bacteria of the Bacillus genus/dose (e.g. B. subtilis and/or B. coagulans).

In one example, the composition described herein comprises at least about 2 mg of dextrin and at least about 76 mg of L-cysteine/dose. For example, the composition described herein may comprise about 2 mg of dextrin/dose and about 76 mg of L-cysteine/dose. This example corresponds with a composition as tested in example 1, test 6B. In each of these examples, the composition may also comprise at least about 300 mg of rice bran/dose (e.g. at least about 300 mg of fermented rice bran/dose) and/or at least 5,000 cfu of bacteria of the Bacillus genus/dose (e.g. B. subtilis and/or B. coagulans). For instance, in each of these examples, the composition may comprise about 300 mg of rice bran/dose (e.g. about 300 mg of fermented rice bran/dose) and/or 5,000 cfu of bacteria of the Bacillus genus/dose (e.g. B. subtilis and/or B. coagulans).

In one example, the composition described herein comprises at least about 2 mg of dextrin/dose and at least about 114 mg of L-cysteine/dose. For example, the composition described herein may comprise about 2 mg of dextrin/dose and about 114 mg of L-cysteine/dose. This example corresponds with a composition as tested in example 1, test 5B. In each of these examples, the composition may also comprise at least about 300 mg of rice bran/dose (e.g. at least about 300 mg of fermented rice bran/dose) and/or at least 5,000 cfu of bacteria of the Bacillus genus/dose (e.g. B. subtilis and/or B. coagulans). For instance, in each of these examples, the composition may comprise about 300 mg of rice bran/dose (e.g. about 300 mg of fermented rice bran/dose) and/or 5,000 cfu of bacteria of the Bacillus genus/dose (e.g. B. subtilis and/or B. coagulans).

In one example, the composition described herein comprises at least about 20 mg of dextrin/dose and at least about 76 mg of L-cysteine/dose. For example, the composition described herein may comprise about 20 mg of dextrin/dose and about 76 mg of L-cysteine/dose. This example corresponds with a composition as tested in example 1, test 8B. In each of these examples, the composition may also comprise at least about 300 mg of rice bran/dose (e.g. at least about 300 mg of fermented rice bran/dose) and/or at least 5,000 cfu of bacteria of the Bacillus genus/dose (e.g. B. subtilis and/or B. coagulans). For instance, in each of these examples, the composition may comprise about 300 mg of rice bran/dose (e.g. about 300 mg of fermented rice bran/dose) and/or 5,000 cfu of bacteria of the Bacillus genus/dose (e.g. B. subtilis and/or B. coagulans).

It is suggested that the high molecular weight low osmolality carbohydrate (e.g.dextrin) and/or L-cysteine create a micro-environment on a cellular level that makes the microbial consortium of the compositions provided herein, upon resuscitation in the intestinal tract, excrete an enzyme cascade targeted towards short carbon chains, e.g. ethanol/alcohol, resulting in preferential targeting of these substrates. As discussed above, generally, about 80% of the intake of alcohol resides in the small intestine for quite some time before being absorbed to the blood. The targeted enzymes act only on the alcohol in the tract and break it down into carbon dioxide and water, bypassing the liver process of converting alcohol into acetaldehyde and acetic acid, i.e. hangover metabolites formed by the liver's conversion of alcohol.

In particular, upon resuscitation of Bacillus cells and their endospores in duodenum and in the small intestine, the cells scan the biochemical conditions of their micro-environment and start to excrete a unique selection of bio-active substances to optimise the conditions, e.g. pH, conductivity, electrolytes, for their survival and multiplication. Nutrients and substrates are essential for survival and subsequent multiplication. When alcohol/ethanol/ethyl alcohol is present in the micro-environment, alcohol-targeted enzymes are excreted to break down alcohol into fragments containing carbon. Carbon that cannot be used by the microbes, or neighbouring tissue cells, as a nutrient will be biochemically metabolised into water and carbon dioxide that can escape the body system without causing any biological consequences/symptoms. It is suggested that high molecular weight low osmolality carbohydrate (e.g. dextrin) and/or L-cysteine enhance this environmental scan and selective excretion of bio-active substances for optimised enzymatic conditions.

As stated elsewhere herein, the alcohol degrading composition described herein may include a cereal (e.g. a cereal grain) such as rice bran. A cereal is any grass cultivated (grown) for the edible components of its grain (botanically, a type of fruit called a caryopsis), composed of endosperm, germ, and bran. The term cereal may also refer to the resulting grain itself (specifically “cereal grain”). Cereal grains are the seeds that come from grasses such as wheat, millet, rice, barley, oats, rye, triticale, sorghum, and maize (corn).

In some examples, the alcohol degrading composition described herein may include a cereal (e.g. a cereal grain) selected from the group consisting of: wheat, millet, rice, barley, oats, rye, triticale, sorghum, and maize (corn). A person of skill in the art would readily be able to identify a suitable cereal (e.g. cereal grain) for use in the composition described herein.

Bran, also known as miller's bran, is the hard outer layers of cereal grain. It comprises aleurone and pericarp. Corn (maize) bran also includes the pedicel (tip cap). Along with germ, bran is an integral part of whole grains, and is often produced as a byproduct of milling in the production of refined grains. Bran is present in cereal grain, including rice, corn (maize), wheat, oats, barley, rye and millet.

Accordingly, in some examples, the alcohol degrading composition described herein may comprise cereal bran. In some examples, the alcohol degrading composition described herein may comprise a cereal bran selected from the group consisting of: rice bran, corn (maize) bran, wheat bran, oat bran, barley bran, rye bran and millet bran.

In some examples, the alcohol degrading composition described herein may comprise oat bran.

In some examples, the alcohol degrading composition described herein may comprise rice bran.

Rice bran is a by-product of the rice milling process. Generally rice milling yields about 15% w/w broken kernels, about 10% w/w rice bran, about 20% hulls and about 55% w/w whole kernels. The composition of rice bran (in percent by weight) is generally 11-13% of water, 18-21% of crude fat and oil, 14-16% crude protein, 8-10% of crude fiber, 9-12% of ash and 33-36% of carbohydrate. Rice bran has naturally occurring lipases that hydrolyze the oil into glycerol and free fatty acids which give the product a rancid smell and taste. As used herein, “rice bran” refers to the hard outer layer of rice which comprises aleurone and pericarp. Along with germ, it is an integral part of whole rice, and, as mentioned above, is often produced as a by-product of milling in the production of refined rice.

The alcohol degrading composition described herein may comprise rice bran in any suitable form. Suitable forms include raw rice bran, recently milled (unhydrolyzed) full-fat rice bran, low-fat rice bran, defatted rice bran, fermented rice bran, stabilized rice bran and so forth. Raw rice bran is rice bran as obtained after milling. Low-fat rice bran and defatted rice bran are derived from full-fat rice bran by solvent extraction or the like. Full-fat rice bran has a fat content of about 14-18% by weight and low fat and defatted rice bran have about 3-14% and less than 3% fat, respectively, on a weight basis.

In one example, the rice bran is formulated as raw rice bran, recently milled (unhydrolyzed) full-fat rice bran, low-fat rice bran, defatted rice bran, fermented rice bran and/or stabilized rice bran.

In another, more preferable, example, the rice bran is formulated as fermented rice bran and/or stabilized rice bran.

In a further example, the rice bran is fermented rice bran. Rice bran undergoes a natural/spontaneous fermentation process by naturally occurring microbial strains, typically soil-bound strains, e.g. Bacillus and Pediococcus.

As used herein, “fermented rice bran” refers to rice bran which has undergone a fermentation process. Fermented rice bran contains probiotic microbes that stabilise the microbiome of the small intestine.

As used herein “stabilized rice bran” refers to rice bran which has been heated for a short period of time, for example by passing it through a high temperature high pressure extruder. The heat stabilizes the rice bran. In other words, “stabilized rice bran” is thus heat treated rice bran. For instance, rice bran may be stabilized after milling is being by heating it at 130 degrees Celsius for less than 10 seconds. In some examples, stabilized rice bran is a dietary fibre. Typically, stabilized rice bran is a dietary fibre that can be catabolized in the colon. As a dietary fibre, stabilised rice bran is a prebiotic interacting with the microbiome of colon.

The microbial contents of fermented rice bran may have a probiotic and stabilising effect on the microbiome of the small intestine and that the rice bran, being a dietary fibre, stabilises the conditions of colon. Another mechanism of action maybe a reduction of intestinal oxidative stress, which normalizes the barrier function of the intestinal mucosa and may result in less alcohol absorption.

Rice bran (e.g. fermented rice bran) is present in the alcohol degrading (e.g. alcohol metabolizing) composition described herein in an appropriate amount or concentration as described below. The amount or concentration of rice bran may therefore be described by reference to the % w/w of composition or by the total weight (e.g. mgs) per dose of composition (in other words the weight per effective dose). As would be clear to a person of skill in the art, and as described in more detail elsewhere herein, a dose may include one or more dosage units (e.g. 2 dosage units). In examples wherein a plurality of dosage units are used to provide an effective dose, the weight per dose corresponds to the total weight of rice bran over the plurality of dosage units.

In one example, the composition comprises about 50% w/w to about 90% w/w of rice bran.

In another example, the composition comprises about 60% w/w to about 80% w/w of rice bran. In another example, the composition comprises about 73% w/w to about 79% w/w of rice bran.

In one example, the composition comprises at least about 50% w/w of rice bran. In another example, the composition comprises at least about 55% w/w of rice bran. In one example, the composition comprises at least about 60% w/w of rice bran. In a further example, the composition comprises at least about 65% w/w of rice bran. In one example, the composition comprises at least about 73% w/w of rice bran. In one example, the composition comprises at least about 75% w/w of rice bran. In one example, the composition comprises at least about 79% w/w of rice bran. In a further example, the composition comprises at least about 85% w/w of rice bran. In one example, the composition comprises at least about 90% w/w of rice bran.

In one example, the composition comprises about 50% w/w of rice bran. In another example, the composition comprises about 55% w/w of rice bran. In one example, the composition comprises about 60% w/w of rice bran. In a further example, the composition comprises about 65% w/w of rice bran. In one example, the composition comprises about 73% w/w of rice bran. In one example, the composition comprises about 75% w/w of rice bran. In one example, the composition comprises about 79% w/w of rice bran. In a further example, the composition comprises about 85% w/w of rice bran. In one example, the composition comprises about 90% w/w of rice bran.

In a particular example, the composition comprises at least about 67% w/w of rice bran.

For instance, the composition may comprise about 67% w/w of rice bran.

In a particular example, the composition comprises at least about 73% w/w of rice bran. For instance, the composition may comprise about 73% w/w of rice bran.

In another example, the composition comprises at least about 79% w/w of rice bran. For instance, the composition may comprise about 79% w/w of rice bran.

In another example, the composition comprises at least about 79.5% w/w of rice bran. For instance, the composition may comprise about 79.5% w/w of rice bran.

In one example, the composition comprises at least about 300 mg of rice bran (e.g. fermented rice bran) per dose. As described elsewhere herein, a dose may be formulated as two capsules.

In one example, the composition comprises at least about 300 mg of rice bran (e.g. fermented rice bran) per dose. In one example, the composition comprises at least about 350 mg of rice bran (e.g. fermented rice bran) per dose. In one example, the composition comprises at least about 400 mg of rice bran (e.g. fermented rice bran) per dose. In one example, the composition comprises at least about 450 mg of rice bran (e.g. fermented rice bran) per dose. In one example, the composition comprises at least about 500 mg of rice bran (e.g. fermented rice bran) per dose. In one example, the composition comprises at least about 550 mg of rice bran (e.g. fermented rice bran) per dose.

In one example, the composition comprises about 300 mg of rice bran (e.g. fermented rice bran) per dose. In one example, the composition comprises about 350 mg of rice bran (e.g. fermented rice bran) per dose. In one example, the composition comprises about 400 mg of rice bran (e.g. fermented rice bran) per dose. In one example, the composition comprises about 450 mg of rice bran (e.g. fermented rice bran) per dose. In one example, the composition comprises about 500 mg of rice bran (e.g. fermented rice bran) per dose. In one example, the composition comprises about 550 mg of rice bran (e.g. fermented rice bran) per dose.

As discussed elsewhere herein, an alcohol degrading composition comprising one or more bacterial species of the Bacillus genus (e.g. B subtilis and/or B. coagulans), rice bran (e.g. fermented rice bran), L-cysteine and a high molecular weight low osmolality carbohydrate (e.g. dextrin) is provided. The one or more bacterial species of the Bacillus genus (e.g. B subtilis and/or B. coagulans), rice bran (e.g. fermented rice bran), L-cysteine and a high molecular weight low osmolality carbohydrate (e.g. dextrin) may be present in the alcohol degrading composition described herein at any appropriate amount or concentration. Appropriate amounts and concentrations of the one or more bacterial species of the Bacillus genus (e.g. B subtilis and/or B. coagulans), rice bran (e.g. fermented rice bran), L-cysteine and the high molecular weight low osmolality carbohydrate (e.g. dextrin) are described elsewhere herein. Illustrative examples of appropriate concentrations are provided below.

In one example, the composition comprises about 0.5% w/w to 5% w/w of dextrin, about 10% w/w of L-cysteine to about 40% w/w of L-cysteine, about 50% w/w to about 90% w/w of rice bran (e.g. fermented rice bran) and about 10,000 cfu/g of bacteria of the Bacillus genus to about 15,000 cfu/g of bacteria of the Bacillus genus (e.g. B. subtilis and/or B. coagulans).

In one example, the composition described herein comprises at least about 0.5% w/w of dextrin, at least about 10% w/w of L-cysteine, at least about 67% w/w of rice bran (e.g. at least about 67% w/w of fermented rice bran) and at least 10,000 cfu/g of bacteria of the Bacillus genus (e.g. B. subtilis and/or B. coagulans).

In one example, the composition described herein comprises at least about 0.5% w/w of dextrin, at least about 20% w/w of L-cysteine, at least about 67% w/w of rice bran (e.g. at least about 67% w/w of fermented rice bran) and at least 10,000 cfu/g of bacteria of the Bacillus genus (e.g. B. subtilis and/or B. coagulans).

In one example, the composition described herein comprises at least about 0.5% w/w of dextrin, at least about 30% w/w of L-cysteine, at least about 67% w/w of rice bran (e.g. at least about 67% w/w of fermented rice bran) and at least 10,000 cfu/g of bacteria of the Bacillus genus (e.g. B. subtilis and/or B. coagulans).

In one example, the composition described herein comprises at least about 5% w/w of dextrin, at least about 20% w/w of L-cysteine, at least about 67% w/w of rice bran (e.g. at least about 67% w/w of fermented rice bran) and at least 10,000 cfu/g of bacteria of the Bacillus genus (e.g. B. subtilis and/or B. coagulans).

In one example, the composition described herein comprises at least about 3% w/w of dextrin, at least about 30% w/w of L-cysteine, at least about 67% w/w of rice bran (e.g. at least about 67% w/w of fermented rice bran) and at least 10,000 cfu/g of bacteria of the Bacillus genus (e.g. B. subtilis and/or B. coagulans).

In one example, the composition comprises about 2 mg to about 50 mg of dextrin/dose, about 38 mg to about 200 mg of L-cysteine/dose, about 300 mg to about 600 mg of rice bran/dose (e.g. fermented rice bran/dose) and about 5,000 cfu of bacteria of the Bacillus genus/dose to about 1×10⁸ cfu of bacteria of the Bacillus genus/dose (e.g. B. subtilis and/or B. coagulans).

In one example, the composition described herein comprises at least about 2 mg of dextrin/dose, at least about 38 mg of L-cysteine/dose, at least about 300 mg of rice bran/dose (e.g. at least about 300 mg of fermented rice bran/dose) and at least 5,000 cfu of bacteria of the Bacillus genus/dose (e.g. B. subtilis and/or B. coagulans).

In one example, the composition described herein comprises at least about 2 mg of dextrin, at least about 76 mg of L-cysteine/dose, at least about 300 mg of rice bran/dose (e.g. at least about 300 mg of fermented rice bran/dose) and at least 5,000 cfu of bacteria of the Bacillus genus/dose (e.g. B. subtilis and/or B. coagulans).

In one example, the composition described herein comprises at least about 2 mg of dextrin/dose, at least about 114 mg of L-cysteine/dose, at least about 300 mg of rice bran/dose (e.g. at least about 300 mg of fermented rice bran/dose) and at least 5,000 cfu of bacteria of the Bacillus genus/dose (e.g. B. subtilis and/or B. coagulans).

In one example, the composition described herein comprises at least about 20 mg of dextrin/dose, at least about 76 mg of L-cysteine/dose, at least about 300 mg of rice bran/dose (e.g. at least about 300 mg of fermented rice bran/dose) and at least 5,000 cfu of bacteria of the Bacillus genus/dose (e.g. B. subtilis and/or B. coagulans).

A composition of 79.5% w/w fermented rice bran (including one or more bacterial species of the Bacillus genus), 0.5% w/w dextrin and 20.0% w/w L-cysteine was assessed to be particularly effective at digesting alcohol in the intestinal tract, having about 50% faster than liver digestion of alcohol, as described in the examples below. Accordingly, in a particular example the composition described herein comprises about 79.5% w/w fermented rice bran (including one or more bacterial species of the Bacillus genus), about 0.5% w/w dextrin and about 20.0% w/w L-cysteine.

The alcohol degrading composition described herein may include one or more additional components e.g. one or more additional ingredients. For example, the composition described herein may further comprise emulsifying agents, filling agents and/or non-active ingredients. A person of skill in the art would readily understand what is meant by “emulsifying agent”, “filling agent” and “non-active ingredient” in the context of the compositions provided herein. Non-limiting examples of additional components include vitamin B12, fatty acid magnesium salts (e.g. magnesium stearate), calcium phosphate, potassium phosphate, silicon dioxide and cellulose (e.g. microcrystalline cellulose).

The additional components described herein may be in any suitable form. Suitable forms would be readily identifiable by a person skilled in the art.

Accordingly, in one example, the alcohol degrading composition described herein may further comprise one or more of: vitamin B12, a fatty acid magnesium salt (e.g. magnesium stearate), calcium phosphate, potassium phosphate, silicon dioxide and cellulose (e.g. microcrystalline cellulose). In another example, the alcohol degrading composition described herein may further comprise two or more of: vitamin B12, a fatty acid magnesium salt (e.g. magnesium stearate), calcium phosphate, potassium phosphate, silicon dioxide and cellulose (e.g. microcrystalline cellulose). In another example, the alcohol degrading composition described herein may further comprise three or more of: vitamin B112, a fatty acid magnesium salt (e.g. magnesium stearate), calcium phosphate, potassium phosphate, silicon dioxide and cellulose (e.g. microcrystalline cellulose). In another example, the alcohol degrading composition described herein may further comprise four or more of: vitamin B112, a fatty acid magnesium salt (e.g. magnesium stearate), calcium phosphate, potassium phosphate, silicon dioxide and cellulose (e.g. microcrystalline cellulose).

In one example, the alcohol degrading composition described further comprises vitamin B12, a fatty acid magnesium salt (e.g. magnesium stearate), calcium phosphate, potassium phosphate, silicon dioxide and cellulose (e.g. microcrystalline cellulose).

Examples of fatty acid magnesium salts include magnesium stearate. Accordingly, in one example, the composition described herein may further comprise magnesium stearate. Magnesium stearate is the magnesium salt of the fatty acid stearic acid. Magnesiums salts of fatty acids (e.g. magnesium stearate) may be an excipient, an inactive ingredient and/or may be used as a lubricant for manufacturing machines. Magnesium stearte is a GRAS-listed ingedient.

In some examples, vitamin B12 is added for regulatory purposes.

In some examples, magnesium salts (e.g. magnesium stearate), calcium salts and/or potassium salts are non-active ingredients (e.g. they have no clinical effects). For example, magnesium salts (e.g. magnesium stearate), calcium salts and/or potassium salts may be added as filling aids (e.g. filling agents) which help capsule filling machines work effectively.

In one example, the composition described herein further comprises microcrystalline cellulose. Microcrystalline cellulose may be used as an emulsifying agent, filling aid and/or a non-active ingredient. Microcrystalline cellulose (and also maltodextrin) may be used as a cake-forming excipient when punching tablets and as a fluidity agent when making capsule formulations. In some examples the composition described herein further comprises maltodextrin.

In one example, the composition comprises about 5% w/w to about 50% w/w of microcrystalline cellulose. In another example, the composition comprises about 5% w/w to about 35% w/w of microcrystalline cellulose. In another example, the composition comprises about 5% w/w to about 20% w/w of microcrystalline cellulose. In another example, the composition comprises about 5% w/w to about 15% w/w of microcrystalline cellulose. In another example, the composition comprises about 5% w/w to about 8% w/w of microcrystalline cellulose.

In one example, the composition comprises at least about 5% w/w of microcrystalline cellulose. In one example, the composition comprises at least about 8% w/w of microcrystalline cellulose. In another example, the composition comprises at least about 10% w/w of microcrystalline cellulose. In another example, the composition comprises at least about 15% w/w of microcrystalline cellulose. In a further example, the composition comprises at least about 20% w/w of microcrystalline cellulose. In another example, the composition comprises at least about 25% w/w of microcrystalline cellulose. In another example, the composition comprises at least about 30% w/w of microcrystalline cellulose. In yet a further example, the composition comprises at least about 35% w/w of microcrystalline cellulose. In another example, the composition comprises at least about 40% w/w of microcrystalline cellulose. In another example, the composition comprises at least about 45% w/w of microcrystalline cellulose. In another example, the composition comprises at least about 50% w/w of microcrystalline cellulose.

In one example, the composition comprises about 5% w/w of microcrystalline cellulose. In one example, the composition comprises about 8% w/w of microcrystalline cellulose. In another example, the composition comprises about 10% w/w of microcrystalline cellulose. In another example, the composition comprises about 15% w/w of microcrystalline cellulose. In a further example, the composition comprises about 20% w/w of microcrystalline cellulose. In another example, the composition comprises about 25% w/w of microcrystalline cellulose. In yet a further example, the composition comprises about 30% w/w of microcrystalline cellulose. In another example, the composition comprises about 35% w/w of microcrystalline cellulose. In another example, the composition comprises about 40% w/w of microcrystalline cellulose. In another example, the composition comprises about 45% w/w of microcrystalline cellulose. In another example, the composition comprises about 50% w/w of microcrystalline cellulose.

In one example, the composition described herein comprises at least about 30 mg of microcrystalline cellulose per dose. As described elsewhere herein, a dose may be formulated in two capsules.

In one example, the composition described herein comprises at least about 40 mg of microcrystalline cellulose per dose. In one example, the composition described herein comprises at least about 50 mg of microcrystalline cellulose per dose. In one example, the composition described herein comprises at least about 60 mg of microcrystalline cellulose per dose. In one example, the composition described herein comprises at least about 70 mg of microcrystalline cellulose per dose. In one example, the composition described herein comprises at least about 80 mg of microcrystalline cellulose per dose. In one example, the composition described herein comprises at least about 90 mg of microcrystalline cellulose per dose. In one example, the composition described herein comprises at least about 100 mg of microcrystalline cellulose per dose.

In one example, the composition described herein further comprises magnesium stearate. Magnesium stearate may be used as an emulsifying agent, filling aid and/or a non-active ingredient.

In one example, the composition comprises about 0.2% w/w to about 1.5% w/w of magnesium stearate. In another example, the composition comprises about 0.5% w/w to about 1.4% w/w of magnesium stearate. In another example, the composition comprises about 0.6% w/w to about 1.3% w/w of magnesium stearate.

In one example, the composition comprises at least about 0.2% w/w of magnesium stearate. In another example, the composition comprises at least about 0.3% w/w of magnesium stearate. In another example, the composition comprises at least about 0.4% w/w of magnesium stearate. In a further example, the composition comprises at least about 0.5% w/w of magnesium stearate. In another example, the composition comprises at least about 0.6% w/w of magnesium stearate. In another example, the composition comprises at least about 0.7% w/w of magnesium stearate. In yet a further example, the composition comprises at least about 0.8% w/w of magnesium stearate. In another example, the composition comprises at least about 0.9% w/w of magnesium stearate. In another example, the composition comprises at least about 1.0% w/w of magnesium stearate. In another example, the composition comprises at least about 1.1% w/w of magnesium stearate. In another example, the composition comprises at least about 1.2% w/w of magnesium stearate. In another example, the composition comprises at least about 1.3% w/w of magnesium stearate. In another example, the composition comprises at least about 1.4% w/w of magnesium stearate. In another example, the composition comprises at least about 1.5% w/w of magnesium stearate.

In one example, the composition comprises about 0.2% w/w of magnesium stearate. In another example, the composition comprises about 0.3% w/w of magnesium stearate. In another example, the composition comprises about 0.4% w/w of magnesium stearate. In a further example, the composition comprises about 0.5% w/w of magnesium stearate. In another example, the composition comprises about 0.6% w/w of magnesium stearate. In another example, the composition comprises about 0.7% w/w of magnesium stearate. In yet a further example, the composition comprises about 0.8% w/w of magnesium stearate. In another example, the composition comprises about 0.9% w/w of magnesium stearate. In another example, the composition comprises about 1.0% w/w of magnesium stearate. In another example, the composition comprises about 1.1% w/w of magnesium stearate. In another example, the composition comprises about 1.2% w/w of magnesium stearate. In another example, the composition comprises about 1.3% w/w of magnesium stearate. In another example, the composition comprises about 1.4% w/w of magnesium stearate. In another example, the composition comprises about 1.5% w/w of magnesium stearate.

In one example, the composition described herein comprises at least about 4 mg of magnesium stearate per dose. As described elsewhere herein, a dose may be formulated in two capsules.

In one example, the composition described herein comprises at least about 5 mg of magnesium stearate per dose. In one example, the composition described herein comprises at least about 6 mg of magnesium stearate per dose. In one example, the composition described herein comprises at least about 7 mg of magnesium stearate per dose. In one example, the composition described herein comprises at least about 8 mg of magnesium stearate per dose. In one example, the composition described herein comprises at least about 9 mg of magnesium stearate per dose. In one example, the composition described herein comprises at least about 10 mg of magnesium stearate per dose.

In one example, the composition described herein further comprises silicon dioxide. Silicon dioxide may be used as an emulsifying agent, filling aid and/or a non-active ingredient.

In one example, the composition comprises about 0.5% w/w to about 4% w/w of silicon dioxide. In another example, the composition comprises about 0.7% w/w to about 3% w/w of silicon dioxide. In another example, the composition comprises about 0.9% w/w to about 2% w/w of silicon dioxide.

In one example, the composition comprises at least about 0.5% w/w of silicon dioxide. In another example, the composition comprises at least about 1% w/w of silicon dioxide. In another example, the composition comprises at least about 1.5% w/w of silicon dioxide. In a further example, the composition comprises at least about 2% w/w of silicon dioxide. In another example, the composition comprises at least about 2.5% w/w of silicon dioxide. In another example, the composition comprises at least about 3% w/w of silicon dioxide. In yet a further example, the composition comprises at least about 3.5% w/w of silicon dioxide. In another example, the composition comprises at least about 4% w/w of silicon dioxide.

In one example, the composition comprises about 0.5% w/w of silicon dioxide. In another example, the composition comprises about 1% w/w of silicon dioxide. In another example, the composition comprises about 1.5% w/w of silicon dioxide. In a further example, the composition comprises about 2% w/w of silicon dioxide. In another example, the composition comprises about 2.5% w/w of silicon dioxide. In another example, the composition comprises about 3% w/w of silicon dioxide. In yet a further example, the composition comprises about 3.5% w/w of silicon dioxide. In another example, the composition comprises about 4% w/w of silicon dioxide.

In one example, the composition described herein comprises at least about 4 mg of silicon dioxide per dose. As described elsewhere herein, a dose may be formulated in two capsules.

In one example, the composition described herein comprises at least about 5 mg of silicon dioxide per dose. In one example, the composition described herein comprises at least about 6 mg of silicon dioxide per dose. In one example, the composition described herein comprises at least about 7 mg of silicon dioxide per dose. In one example, the composition described herein comprises at least about 8 mg of silicon dioxide per dose.

In one example, the composition described herein further comprises vitamin B12. Vitamin B12 may be included in the composition described herein for regulatory purposes. In one example, the composition comprises at least 15% of recommended daily intake (RDI), i.e. 0.38 mcg up to 2.4 mcg (EU) and 2.5 mcg for USA.

In one example, the composition described herein comprises at least about 0.76 μg (mcg) of vitamin B112 per dose. In one example, the composition described herein comprises at least about 0.9 μg (mcg) of vitamin B12 per dose.

The composition may be formulated in any appropriate form. For example, it may be in the form of a tablet or capsule. In one example, the composition is formulated as an acid resistant tablet or capsule. Generally, “capsule” refers to both empty and filled capsules whereas “shell” specifically refers to an empty capsule unless the context requires otherwise.

In some examples, the composition described herein is comprised within a capsule (e.g. within a shell). In some examples, the composition described herein is comprised within an acid resistant capsule (e.g. within an acid resistant shell). As would be clear to a person of skill in the art, when reference is made to a % w/w of an ingredient present within the composition, this does not take into account any weight attributed to the capsule (and thus only takes into account the % w/w of the composition within the capsule).

In one example, the acid resistant tablet or capsule comprises a film coating, wherein the film coating comprises hydroxypropyl methylcellulose (HPMC). HPMC is a semisynthetic, inert, viscoelastic polymer used in various applications. For instance, HPMC may be used as an excipient in oral tablet and capsule formulations, where, depending on the grade, it functions as controlled release agent to delay the release of a medicinal compound into the digestive tract. In tablets, HPMC may also be used as a binder and/or as a component of tablet coatings.

In one example, the composition described herein is comprised within a (capsule) shell wherein the shell comprises hydroxypropyl methylcellulose (HPMC).

A person of skill in the art would understand the meaning of “acid resistant” in the context of the present invention, particularly within the context of an ingestible composition as described herein. For instance, the composition described herein may be formulated as an acid resistant tablet or capsule or may be comprised within an acid resistant capsule (e.g. an acid resistant shell) which is dissolved once reaching the duodenum, allowing the microbial cells and spores to be released and settle in the upper part of the intestinal tract.

In one example, the composition present within a capsule, wherein the capsule comprises about 80 mg to about 100 mg of HPMC. For example, the capsule may comprise at least about 80 mg of HPMC. In one example, the capsule comprises at least about 90 mg of HPMC. In one example, the capsule comprises at least about 100 mg of HPMC. In one example, the capsule comprises about 80 mg of HPMC. In one example, the capsule comprises about 90 mg of HPMC. In another example, the capsule comprises about 100 mg of HPMC.

Typically HPMC is present as a film coating at the exterior surface of the capsule described herein.

The composition described herein may be in unit dosage form. Where the composition described herein is in unit dosage form, it may be that one tablet or capsule is administered and this constitutes a dose, alternatively it may be that two tablets or capsules are administered and this constitutes a dose. Suitable dosages and regimens can be determined by a person skilled in the art, based on the examples below. Accordingly, in one example, a dose of the composition according to the present invention includes a plurality of small tablets or capsules (e.g. two).

The term “dosage form” as used herein, refers to an amount of medication to be taken at one time, optionally in regular intervals. This is also referred to as a “dose” herein.

In an example, the invention provides a solid unit dose form for oral administration.

In a particular example, a dose of the composition described herein comprises: about 300 mg of fermented rice bran, about 38 mg of L-cysteine, about 30 mg of microcrystalline cellulose, about 4 mg of magnesium stearate, about 4 mg of silicon dioxide, about 2 mg of dextrin, about 0.76 μg (mcg) vitamin B12 and about 5,000 cfu bacteria from the Bacillus genus. In this particular example, the dose may be formulated as two tablets or capsules. In other words, the amounts of the components may represent a total amount of the component present in the dose (i.e. in the two tablets or capsules). This corresponds to the composition that is used in Example 1, test 7B. A skilled person would thus understand that at least each of these components, in at least these amounts, provide an effective dose in accordance with the invention. Accordingly, in one example, dose of the composition described herein comprises: at least about 300 mg of fermented rice bran, at least about 38 mg of L-cysteine, at least about 30 mg of microcrystalline cellulose, at least about 4 mg of magnesium stearate, at least about 4 mg of silicon dioxide, at least about 2 mg of dextrin, at least about 0.76 μg (mcg) vitamin B112 and at least about 5,000 cfu bacteria from the Bacillus genus.

In a particular example, a dose of the composition described herein comprises: about 552 mg of fermented rice bran, about 150 mg of L-cysteine, about 40 mg of microcrystalline cellulose, about 4.8 mg of magnesium stearate, about 4 mg of silicon dioxide, about 4 mg of dextrin, about 0.9 μg (mcg) vitamin B12 and about 100,000 cfu bacteria from the Bacillus genus. In this particular example, the dose may be formulated as two tablets or capsules. In other words, the amounts of the components may represent a total amount of the component present in the dose (i.e. in the two tablets or capsules). A skilled person would thus understand that at least each of these components, in at least these amounts, provide an effective dose in accordance with the invention. Accordingly, in one example, dose of the composition described herein comprises: at least about 552 mg of fermented rice bran, at least about 150 mg of L-cysteine, at least about 40 mg of microcrystalline cellulose, at least about 4.8 mg of magnesium stearate, at least about 4 mg of silicon dioxide, at least about 4 mg of dextrin, at least about 0.9 μg (mcg) vitamin B12 and at least about 100,000 cfu bacteria from the Bacillus genus.

Typically HPMC is present as a film coating at the exterior surface of the capsule described herein.

The composition described herein may be used as (or used as part of) a dietary supplement, a nutraceutical, a food composition, a medical food, or a medicament.

The term “dietary supplement” or “food supplement” as used herein, refers to a composition which is consumed in addition to the daily meals or in between.

The term “food composition” as used herein, refers to any kind of composition which is eatable and/or drinkable without causing toxic symptoms in the subject eating or drinking the respective composition.

Accordingly, the use of a composition described herein for degrading alcohol is provided herein. The term “degrading alcohol” is described elsewhere herein and applies equally here.

The composition may be used for degrading alcohol in a subject (e.g. the use may be in vivo). The subject may be any suitable subject, for example, the subject may be human. The subject may be a human that is intending to consume or has consumed alcohol.

In one example, the composition may be used for metabolising alcohol in the gut of the subject. The term “metabolising alcohol” is described elsewhere herein and applies equally here. In one example, the composition may be used for metabolising alcohol in the intestine of the subject. In a particular example, the composition may be used for metabolising alcohol in the small intestine of the subject. More specifically, the composition may be used for metabolising alcohol in the duodenum of the subject.

In one example, the composition may be used for reducing absorption of alcohol into the blood of the subject. A person of skill in the art would be able to determine a reduction in absorption of alcohol into the blood in the presence of the composition provided herein (compared to when the composition is not used) using methods known in the art (e.g. the methods described in the examples section below).

When the composition is used to degrade alcohol in a subject, it may be used for reducing breath or blood alcohol concentration in the subject. A person of skill in the art would be able to determine a reduction in breath or blood alcohol concentration in the presence of the composition provided herein (compared to when the composition is not used) using methods known in the art (e.g. the methods described in the examples section below). For instance, a person of skill in the art would readily be able to determine a reduction in breath or blood alcohol concentration in the presence of the composition provided herein (compared to when the composition is not used) using methods known in the art (e.g. the methods described in the examples section below) about 30 minutes after alcohol ingestion (alcohol consumption).

### Use of L-Cysteine for Increasing Alcohol Degradation

The use of L-cysteine for increasing alcohol degradation by one or more bacterial species of the Bacillus genus is provided herein. L-cysteine may be used in vitro or in vivo for increasing alcohol degradation. For example, L-cysteine may be used for increasing alcohol degradation by one or more bacterial species of the Bacillus genus when it is used as (or used as part of) a dietary supplement, a nutraceutical, a medical food or a medicament.

The terms “L-cysteine”, “alcohol degradation” and “one or more bacterial species of the Bacillus genus” are defined elsewhere herein, with examples of what is encompassed by these terms. These definitions and aspects apply equally here.

A person of skill in the art would be able to determine increased alcohol degradation in the presence of L-cysteine (compared to when L-cysteine is not used) using methods known in the art (e.g. the methods described in the examples section below).

In one specific example, L-cysteine may be used for increasing alcohol degradation by one or more bacterial species of the Bacillus genus selected from B. subtilis and B. coagulans. As described elsewhere herein, the one or more bacterial species of the Bacillus genus may comprise B. subtilis. Alternatively, the one or more bacterial species of the Bacillus genus may comprise B. coagulans. In one example, the one or more bacterial species of the Bacillus genus may comprise B. subtilis and B. coagulans. Features of these bacterial species are provided elsewhere herein, which apply equally to this aspect.

Suitable concentrations, amounts, proportions etc of the one or more bacterial species of the Bacillus genus are described elsewhere herein and apply equally to this aspect.

In one specific example, L-cysteine may be used for increasing alcohol degradation by one or more bacterial species of the Bacillus genus, when the alcohol is ethyl alcohol.

In one example, the L-cysteine may be combined with a high molecular weight low osmolality carbohydrate, such as dextrin, when it is used for increasing alcohol degradation by one or more bacterial species of the Bacillus genus. In addition, or alternatively, the L-cysteine may be combined with rice bran.

In one example, L-cysteine may be used for increasing alcohol degradation by one or more bacterial species of the Bacillus genus when it is used as (or used as part of) a dietary supplement, a nutraceutical or a medical food (e.g. when it is part of a composition that is described herein).

### Methods of Treatments and Uses

As described elsewhere herein, the composition described herein may be used in vitro or in vivo for degrading alcohol. The compositions described herein may therefore be used as a medicament.

In one example, the compositions described herein may be used as a medicament for degrading alcohol in a subject. The compositions provided herein may be particularly advantageous, for example, for preventing and/or treating alcohol-induced organ damage in a subject. For example, the compositions provided herein may be useful for preventing and/or treating alcohol-induced damage to the liver and/or pancreas of a subject.

“Alcohol induced organ damage” as used herein includes both acute and chronic alcohol induced organ damage. Typically, alcohol induced damage comprises inflammation and/or inflammatory conditions which cause malfunction of internal organs and tissues, leading to severe secondary and sometimes life-threatening diseases. The liver sustains the greatest degree of tissue injury by heavy drinking because it is the primary site of ethanol metabolism. For instance, chronic and excessive alcohol consumption produces a wide spectrum of hepatic lesions, the most characteristic of which are steatosis, hepatitis, and fibrosis/cirrhosis. Steatosis is the earliest response to heavy drinking and is characterized by the deposition of fat in hepatocytes. Steatosis can progress to steatohepatitis, which is a more severe, inflammatory type of liver injury. This stage of liver disease can lead to the development of fibrosis, during which there is excessive deposition of extracellular matrix proteins. The fibrotic response begins with active pericellular fibrosis, which may progress to cirrhosis, characterized by excessive liver scarring, vascular alterations, and eventual liver failure.

Other examples of alcohol induced damage include both acute and chronic pancreatitis. Pancreatitis is defined as inflammation of the pancreas, leading to damage and dysfunction of the retroperitoneal organ. There are various etiologies of pancreatitis, the most common being alcohol and gallstones. Acute pancreatitis (AP) is a necro-inflammatory disease resulting from exocrine cell destruction by infiltrating inflammatory cells. The diagnostic criteria are typically when a patient presents with characteristic symptoms, elevated lipase levels, and distinct imaging findings. Acute pancreatitis will either resolve with the pancreas fully regenerating, lead to transient organ failure, or progress to cause systemic inflammation and multi-organ failure. Chronic pancreatitis (CP) is believed to result from recurrent attacks of acute pancreatitis, leading to the development of pancreatic insufficiency, steatorrhea, diabetes, pancreatic calcification, and fibrosis. Alcohol predisposes the pancreas to damage from otherwise benign agents. As a result, one of the main strategies to prevent recurrent attacks involve providing alcohol (and smoking) cessation counseling and strategies to patients.

The compositions provided herein may be useful for preventing and/or treating a disease, condition or illness selected from the group consisting of: alcohol induced fatty liver, alcohol induced hepatitis, liver cirrhosis, alcohol induced cancer, cardio-vascular conditions, obesity, neuropathy, neurodegenerative diseases, hangover symptoms, flushing syndrome, headache and/or intoxication by acetaldehyde. In one example, the compositions provided herein may be useful for preventing and/or treating a disease, condition or illness selected from the group consisting of: alcohol induced fatty liver, alcohol induced hepatitis, liver cirrhosis, and alcohol induced cancer.

Alcohol induced fatty liver is also referred to herein as alcoholic fatty liver. Similarly, alcohol induced hepatitis is also referred to herein as alcoholic hepatitis.

Examples of alcohol induced cancers are well known in the art, and include liver cancer, pancreatic cancer, breast cancer, esophageal cancer and oropharyngolaryngeal cancer. The term “oropharyngolaryngeal cancer” as used herein, refers to cancers derived from oral cavity, pharynx, larynx or upper esophagus. At early stage of the cancer of this area, the origin could be determined. However, very often, the cancer of this area is found at the late invasive stage and the origin cannot be determined. Therefore, cancers arising from this sources are collectively termed “oropharyngolaryngeal cancer”. A person skilled in the art would readily be able to identify cancers associated with different parts of the body as described above.

The term “neuropathy” as used herein, refers to any disease or abnormality of the neurons of the nervous system. “In particular “neuropathy” means a disorder of the peripheral nervous system, affecting nerves anywhere except brain and the spinal cord. A non-limiting example for neuropathy is alcoholic polyneuropahy which is characterized by numbness, abnormal sensations called dysesthesias and allodynias that occur either spontaneously or in reaction to external stimuli, and a characteristic form of pain, called neuropathic pain or neuralgia.

The term “neurodegenerative disease” as used herein, refers to any disease or abnormality of the of the nervous system caused by deterioration of neurons, which include death of neurons and functional loss of neurotransmitters. Non-limiting examples for a neurodegenerative disease are Alzheimer's disease (e.g. late-onset Alzheimer's Disease) and Parkinson's disease. The term “late-onset Alzheimer's Disease” as used herein, refers to the onset of Alzheimer's Disease in elderly people, in particular in people being 65 years old and older.

In one example, the neurodegenerative disease is Alzheimer's Disease, optionally the neurodegenerative disease is late-onset Alzheimer's Disease.

As used herein, “hangover” refers to general discomfort generally suffered on waking by a person who has drunk (e.g. consumed and/or ingested) alcohol (to excess). In other words, hangover is the result of intoxication of the organism, caused by the ingestion of (an excessive amount) of alcohol. Hangover is the appearance of a series of symptoms on the day after having drunk alcohol excessively and can become worse if the person smokes excessively. The organism protects itself from intoxication and secretes enzymes that metabolise and excrete the toxins. However, when the ingestion of alcohol is excessive the capacity of the organism to metabolise it is less and the symptoms of hangover appear. “Hangover symptoms” include a headache, produced by dilation of blood vessels caused by the accumulation of histamine, red eyes, occasional memory loss, vomiting, possible flatulence, intense thirst, which originates as a response of the body to dehydration caused by the alcohol, abdominal and muscular pain, which results in a feeling of weakness and in some cases, diarrhoea. In particular, headache is common in all people suffering from hangover and results from dilation of the blood vessels due to the effect of some vasodilator substances (such as histamine) in the person.

The term “flushing syndrome,” as used herein, refers to the flushing as a consequence of drinking alcohol. Flushing is associated with the erythema (reddening caused by dilation of capillaries) of the face, neck, and shoulder, after consumption of alcohol. Flushing after alcohol consumption is often associated with a range of symptoms: dizziness, nausea, headaches, an increased pulse, occasional extreme drowsiness, and occasional skin swelling and itchiness. There symptoms are collectively called “Flushing syndrome” or “Asian flush”.

The compositions provided herein are able to degrade alcohol in the gut (intestine) of a subject, thereby reducing the amount of alcohol that is absorbed into the blood. The compositions provided herein can therefore degrade alcohol before the alcohol is transported to the liver. The compositions provided herein can therefore be used to protect the liver and associated organs from alcohol induced liver damage.

The compositions provided herein are effective at degrading alcohol when they are administered to a subject at any point before, during (e.g. simultaneously with alcohol ingestion or between ingestion of alcohol e.g. between a first alcoholic drink and a second alcoholic drink), or after ingestion of alcohol. From the viewpoint of effects, the agent is preferably ingested before or during ingestion of alcohol, most preferably at least one hour before ingestion of alcohol. In some examples, the composition is for administration before or simultaneously with alcohol ingestion.

In some examples, the composition (e.g. an effective amount of the composition described herein) is ingested at least five hours before ingestion of alcohol. In some examples, the composition (e.g. an effective amount of the composition described herein) is ingested at least four hours before ingestion of alcohol. In some examples, the composition (e.g. an effective amount of the composition described herein) is ingested at least three hours before ingestion of alcohol. In some examples, the composition (e.g. an effective amount of the composition described herein) is ingested at least two hours before ingestion of alcohol.

In one example, the composition (e.g. an effective amount of the composition described herein) is ingested at least one hour before ingestion of alcohol.

In one example, the composition (e.g. an effective amount of the composition described herein) is ingested at least 30 minutes before ingestion of alcohol.

In one example, the composition (e.g. an effective amount of the composition described herein) is ingested at least 15 minutes before ingestion of alcohol.

In case of using AB001 to protect from alcohol ingested by occasional drinking, it may be beneficial to take two doses prior to drinking with a 2-hour time difference between them, and the last dose taken at least 2 hours prior to the ingestion of alcohol.

In some examples, the composition (e.g. an effective amount of the composition described herein) is ingested at least 1 day before ingestion of alcohol.

In one example, the composition described herein (e.g. a dose of the composition described herein) is taken daily. In some examples, the composition (e.g. an effective amount of the composition described herein) is ingested daily for at least 2 days, at least 3 days, at least 4 days, at least 5 days, at least 6 day or at least 7 days before ingestion of alcohol. For instance, one dose (e.g. 2 capsules or tablets of the composition described herein) may be taken daily for at least 2 days, at least 3 days, at least 4 days, at least 5 days, at least 6 days or at least 7 days preceding alcohol ingestion. For instance, one dose (e.g. 2 capsules or tablets of the composition described herein) may be taken daily for 2 days, 3 days, 4 days, 5 days, 6 days or 7 days preceding alcohol ingestion.

In a particular example, one dose (e.g. 2 capsules or tablets of the composition described herein) is taken daily for 7 days preceding alcohol ingestion. In other words, the composition described herein (e.g. an effective amount of the composition described herein, such as a dose of the composition described herein) is taken daily for 1 week prior to alcohol ingestion.

In one example, the composition (e.g. an effective amount of the composition described herein) is ingested at least 15 minutes before ingestion of alcohol.

In some examples, the composition (e.g. an effective amount of the composition described herein) is ingested simultaneously with alcohol. In other words, the composition (e.g. an effective amount of the composition described herein) is ingested at the same time as alcohol.

In other examples, the composition (e.g. an effective amount of the composition described herein) is ingested after (e.g. following) alcohol ingestion. For instance, the composition (e.g. an effective amount of the composition described herein) may be ingested after a first alcoholic beverage has been ingested. In another example, the composition (e.g. an effective amount of the composition described herein) may be ingested after a first alcoholic beverage has been ingested but before a second alcoholic beverage is ingested and so on, for instance, the composition (e.g. an effective amount of the composition described herein) may be ingested after a second alcoholic beverage has been ingested but before a third alcoholic beverage is ingested.

In some examples, the composition (e.g. an effective amount of the composition described herein) is ingested at least five hours after ingestion of alcohol. In some examples, the composition (e.g. an effective amount of the composition described herein) is ingested at least four hours after ingestion of alcohol. In some examples, the composition (e.g. an effective amount of the composition described herein) is ingested at least three hours after ingestion of alcohol. In some examples, the composition (e.g. an effective amount of the composition described herein) is ingested at least two hours after ingestion of alcohol.

In one example, the composition (e.g. an effective amount of the composition described herein) is ingested at least one hour after ingestion of alcohol.

In one example, the composition (e.g. an effective amount of the composition described herein) is ingested at least 30 minutes after ingestion of alcohol.

In one example, the composition (e.g. an effective amount of the composition described herein) is ingested at least 15 minutes after ingestion of alcohol.

It would be clear to the skilled person that the composition described herein may be administered to a subject at any point before, during, or after ingestion of alcohol. As described above, from the viewpoint of effects, the agent is preferably ingested before or during ingestion of alcohol, most preferably at least one hour before ingestion of alcohol.

“Ingestion” as used herein, refers to the taking in (e.g. consumption) of a substance by an organism. Accordingly, as used herein, “alcohol ingestion” refers to the act or process of taking alcohol into an organism (e.g. a subject, preferably a human). In the context of the present invention, alcohol is preferably ingested via the mouth where the subject is human. “Alcohol ingestion” and “alcohol consumption” may be used interchangeably herein.

The composition may be orally ingested in the form of an aqueous solution, a tablet, a capsule, a granule, or the like. Moreover, the composition may be added to an alcoholic beverage (or other source of alcohol) before it is ingested. For example, the composition may be added as an additional ingredient during the production of an alcoholic beverage.

An appropriate ingestion amount of the composition disclosed herein depends on the mass of alcohol that is (to be) ingested.

A pharmaceutical formulation is also provided herein, comprising the composition of the invention. As would be clear to a person of skill in the art, a pharmaceutical formulation is a formulation that is suitable for administration to a subject for degrading alcohol. The pharmaceutical formulation may be for administration to a subject for preventing and/or treating a disease, condition or illness selected from the group consisting of: alcohol induced fatty liver, alcohol induced hepatitis, liver cirrhosis, alcohol induced cancer, cardio-vascular conditions, obesity, neuropathy, neurodegenerative diseases, hangover symptoms, flushing syndrome, headache and/or intoxication by acetaldehyde. A pharmaceutical formulation, as used herein, comprises an effective dose of the composition of the invention.

The compositions described herein are for administration to a subject (preferably a human) in an effective amount (in an effective dose). An “effective amount” is an amount that alone, or together with further doses, produces the desired (therapeutic or non-therapeutic) response. The effective amount to be used will depend, for example, upon the therapeutic (or non-therapeutic) objectives, the route of administration, and the condition of the subject. For example, a suitable dosage of the composition of the invention for a given subject can be determined by a physician (or the person administering the composition), taking into consideration various factors known to modify the action of the composition of the invention for example amount of alcohol ingestion, body weight, sex, diet, time and route of administration, other medications and other relevant clinical factors. The dosages and schedules may be varied according to the particular condition, disorder or symptom the overall condition of the subject. Effective dosages may be determined by either in vitro or in vivo methods.

The compositions described herein are advantageously presented in dosage units. For example, the composition may be presented in the form of a capsule or a tablet. Other suitable dosage units are described elsewhere herein.

An “effective amount” may comprise administration of one or more dosage units. For example, an effective amount may be achieved by administration of one or two capsules or tablets. When the effective amount comprises a plurality of dosage units, the dosage units may be administered together, or they may be taken at spaced intervals during the day.

Alternative appropriate effective amounts and dosage forms will be readily identifiable by a person of skill in the art, based on the examples below, using routine experimentation.

As used herein, the terms “treating” and “treatment” refer to the administration of a composition to a subject (e.g., a symptomatic subject afflicted with an adverse condition, disorder, illness or disease) so as to affect a reduction in severity and/or frequency of a symptom, eliminate a symptom and/or its underlying cause, and/or facilitate improvement or remediation of damage, and/or preventing an adverse condition, disorder, illness or disease in an asymptomatic subject who is susceptible to a particular adverse condition, disorder, illness or disease, or who is suspected of developing or at risk of developing the condition, disorder, illness or disease.

The term “prevention” as used herein means the avoidance of the occurrence or re-occurrence a symptom and/or its underlying cause, damage, an adverse condition, disorder, illness and/or disease. For instance, “prevention” in the context of the present invention may be avoiding the appearance of one or more of the symptoms related to hangover.

As discussed elsewhere herein, the subject may be any suitable subject, for example, the subject may be human. The subject may be a human that is intending to consume or has consumed alcohol.

As used herein, a “pharmaceutical composition” refers to a composition having pharmacological activity or other direct effect in the mitigation, treatment, or prevention of disease, and/or a finished dosage form or formulation thereof and is for human use. A pharmaceutical composition or pharmaceutical preparation is typically produced under good manufacturing practices (GMP) conditions. Pharmaceutical compositions or preparations may be sterile or non-sterile. If non-sterile, such pharmaceutical compositions or preparations typically meet the microbiological specifications and criteria for non-sterile pharmaceutical products as described in the U.S. Pharmacopeia (USP) or European Pharmacopoeia (EP). Accordingly, the composition described herein may be formulated as a pharmaceutical composition. In some examples, the pharmaceutical composition is non-sterile.

Unless defined otherwise herein, all technical and scientific terms used herein have the same meaning as commonly understood by one of ordinary skill in the art to which this invention pertains. For example, Singleton and Sainsbury, Dictionary of Microbiology and Molecular Biology, 2d Ed., John Wiley and Sons, NY (1994); and Hale and Marham, The Harper Collins Dictionary of Biology, Harper Perennial, NY (1991) provide those of skill in the art with a general dictionary of many of the terms used in the invention. Although any methods and materials similar or equivalent to those described herein find use in the practice of the present invention, the preferred methods and materials are described herein. Accordingly, the terms defined immediately below are more fully described by reference to the Specification as a whole. Also, as used herein, the singular terms “a”, “an,” and “the” include the plural reference unless the context clearly indicates otherwise. Unless otherwise indicated, nucleic acids are written left to right in 5′ to 3′ orientation; amino acid sequences are written left to right in amino to carboxy orientation, respectively. It is to be understood that this invention is not limited to the particular methodology, protocols, and reagents described, as these may vary, depending upon the context they are used by those of skill in the art.

Aspects of the invention are demonstrated by the following non-limiting examples.

### EXAMPLES

### Example 1—Development of the composition described herein

### Background

The inventors have previously developed a food grade quality of fermented rice bran for evaluation purposes. A safety study was initiated applying a case study design. Around 1,400 people have taken the product on a regular long-term basis. No side effects/adverse reactions have been reported. However, indications of anti-hangover effects were reported. The inventors sought to investigate this effect further.

### Experiments

A test was organised to confirm the anti-hangover effects of fermented rice bran in a controlled way. Eight individuals, men in their 30s to 50s, had four pints of beer each (4.2%) in one and a half hours at a first occasion. Average 1.24‰30 minutes post drinking and 5.5 hours to ≤0.20‰.

Two weeks later the same group went through the identical procedure but taking 0.75 g of a powder of fermented rice bran pre-drinking. Average 0.91‰30 minutes post drinking and ≤0.20‰4.0 hours post drinking. 25% faster than liver digestion of alcohol. Measurements were made by Breathalyser.

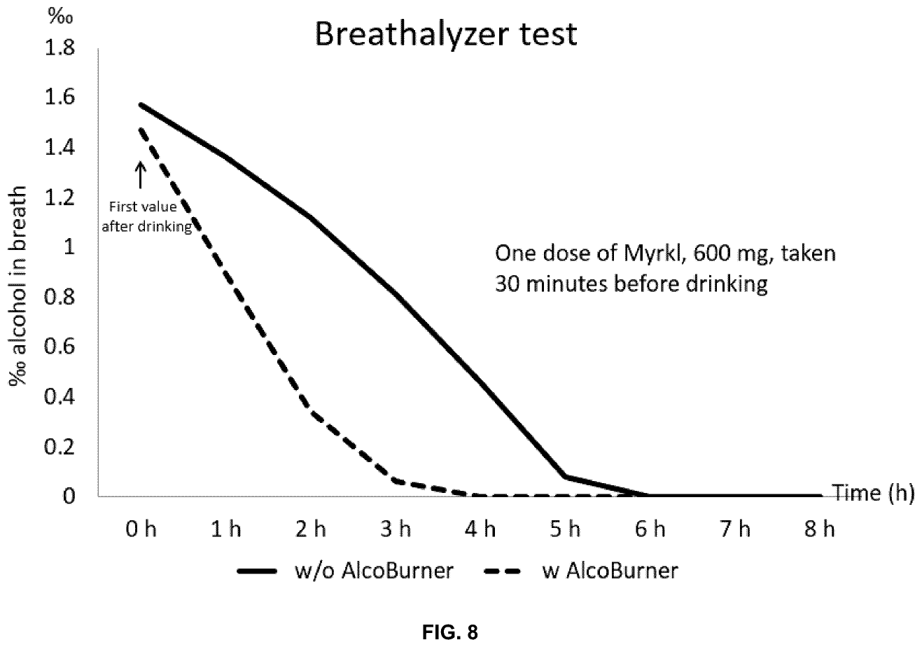

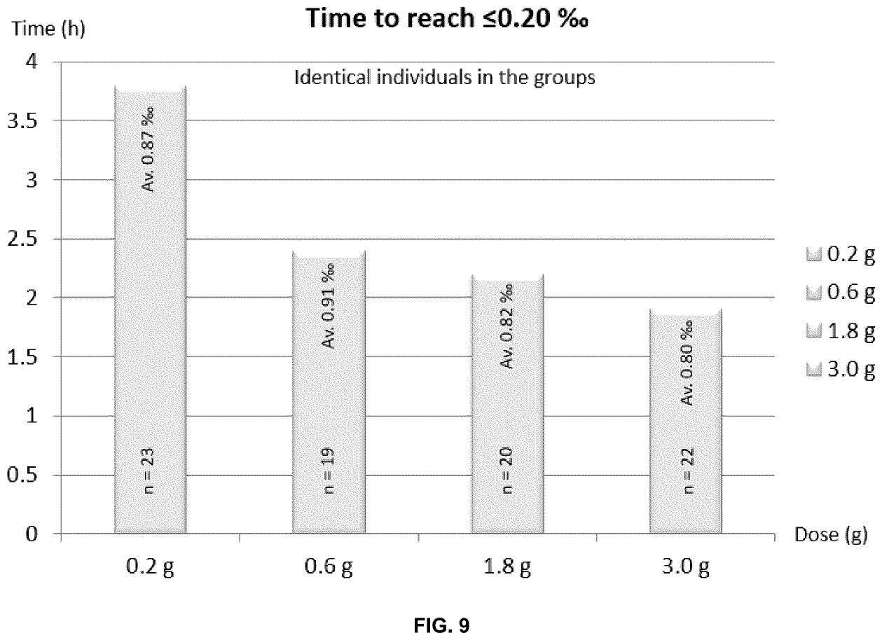

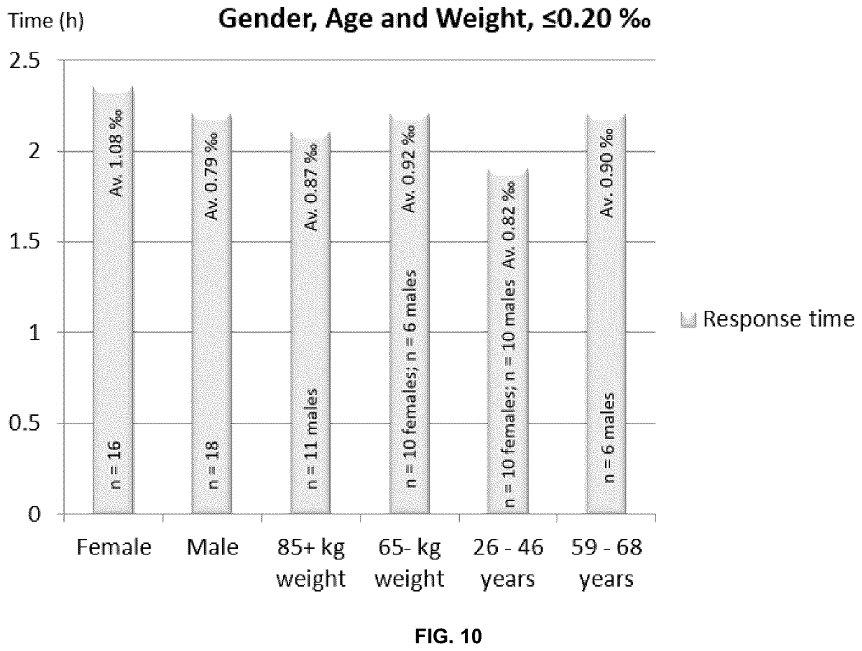

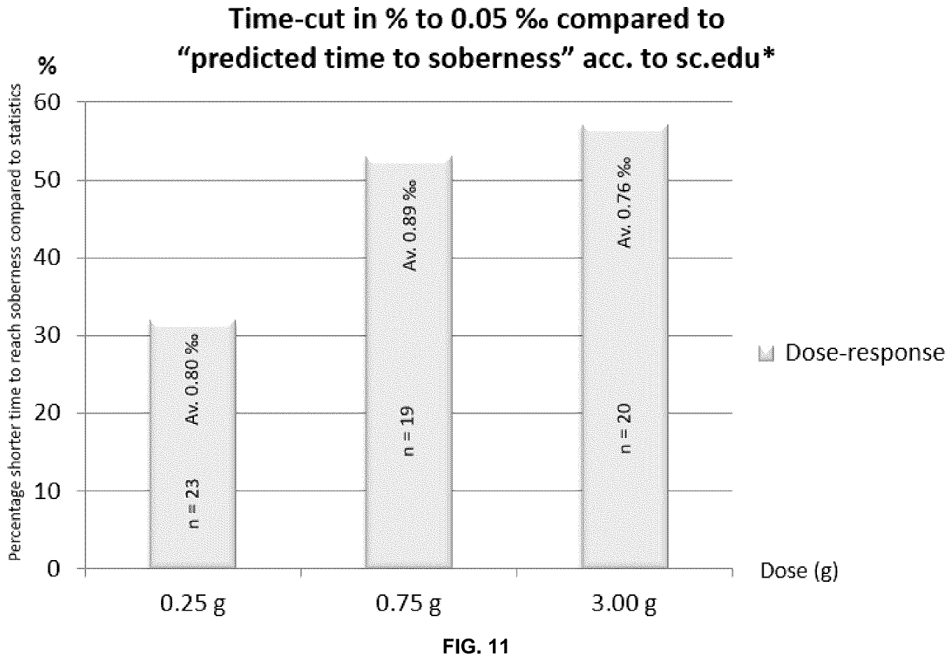

More comprehensive tests were then conducted including both genders, and participants with a wider age and weight span. All tests were of cross-over design, i.e. same group without and with fermented rice bran (referred to herein as “Pinch”, note that “Pinch”, as used herein, may further comprise other components, such as dextrin and/or L-cysteine as described below) at two time-separated occasions, using either iBAC or Drager breathalysers. Participants were offered tea/coffee and a bread roll with cheese before drinking 60 cl of red wine, 13,5%, in four hours. Breathalyser measurements were taken every hour starting 15 minutes post drinking with endpoint ≤0.20‰. The results of the tests are shown below. The composition of fermented rice bran powder used for each test is also described below. The data is summarised in FIGS. 8 to 11.

### Test 1

### Test 1a

16 participants, male 11 and 5 female, age 24 to 68, weight 46 to 92 kg without Pinch
15 minutes: average 0.97‰
1 hour: average 0.89‰
2 hours: average 0.52‰
3 hours: average 0.42‰
4 hours: average 0.29‰
5 hours: average 0.16‰

### Test 1b, Pinch=Fermented Rice Bran Only

13 participants, male 9 and 4 female, age 24 to 68, weight 46 to 92 kg with Pinch
15 minutes: average 1.03‰
1 hour: average 0.78‰
2 hours: average 0.41‰
3 hours: average 0.22‰
4 hours: average 0.07‰

### Test 2

### Test 2a

14 participants, male 10 and 4 female, age 22 to 62, weight 46 to 87 kg without Pinch
15 minutes: average 0.91‰
1 hour: average 0.80‰
2 hours: average 0.52‰
3 hours: average 0.44‰
4 hours: average 0.24‰
5 hours: average 0.11‰

### Test 2b, Pinch=Fermented Rice Bran+5% Dextrin

14 participants, male 10 and 4 female, age 22 to 62, weight 46 to 87 kg with Pinch
15 minutes: average 0.93‰
1 hour: average 0.72‰
2 hours: average 0.36‰
3 hours: average 0.19‰
4 hours: average 0.03

### Test 3

### Test 3a

17 participants, male 12 and 5 female, age 20 to 59, weight 42 to 97 kg without Pinch
15 minutes: average 1.04‰
1 hour: average 0.90‰
2 hours: average 0.61‰
3 hours: average 0.42‰
4 hours: average 0.21‰
5 hours: average 0.11‰

### Test 3b, Pinch=Fermented Rice Bran+2% Dextrin

13 participants, male 9 and 4 female, age 20 to 48, weight 46 to 84 kg with Pinch
15 minutes: average 0.96‰
1 hour: average 0.76‰
2 hours: average 0.34‰
3 hours: average 0.18‰
4 hours: average 0.08‰

### Test 4

### Test 4a

18 participants, male 11 and 7 female, age 20 to 69, weight 42 to 125 kg without Pinch
15 minutes: average 0.96‰
1 hour: average 0.87‰
2 hours: average 0.62‰
3 hours: average 0.39‰
4 hours: average 0.20‰
5 hours: average 0.08‰

### Test 4b, Pinch=Fermented Rice Bran+0.5% Dextrin

15 participants, male 10 and 5 female, age 24 to 64, weight 42 to 125 kg with Pinch
15 minutes: average 0.96‰
1 hour: average 0.76‰
2 hours: average 0.35‰
3 hours: average 0.16‰
4 hours: average 0.04‰

### Test 5

### Test 5a

17 participants, male 11 and 6 female, age 28 to 64, weight 43 to 125 kg without Pinch
15 minutes: average 0.92‰
1 hour: average 0.81‰
2 hours: average 0.65‰
3 hours: average 0.44‰
4 hours: average 0.23‰
5 hours: average 0.06‰

### Test 5b, Pinch=Fermented Rice Bran+0.5% Dextrin+30% L-Cysteine

16 participants, male 11 and 5 female, age 28 to 64, weight 43 to 125 kg with Pinch
15 minutes: average 0.98‰
1 hour: average 0.71‰
2 hours: average 0.32‰
3 hours: average 0.05‰

### Test 6

### Test 6a

14 participants, male 8 and 6 female, age 22 to 69, weight 41 to 125 kg without Pinch
15 minutes: average 0.92‰
1 hour: average 0.84‰
2 hours: average 0.61‰
3 hours: average 0.42‰
4 hours: average 0.27‰
5 hours: average 0.08‰

### Test 6b, Pinch=Fermented Rice Bran+0.5% Dextrin+20% L-Cysteine

16 participants, male 11 and 5 female, age 28 to 62, weight 43 to 125 kg with Pinch
15 minutes: average 0.98‰
1 hour: average 0.65‰
2 hours: average 0.28‰
3 hours: average 0.06‰

### Test 7

### Test 7a

16 participants, male 8 and 8 female, age 21 to 65, weight 42 to 125 kg without Pinch
15 minutes: average 1.03‰
1 hour: average 0.90‰
2 hours: average 0.73‰
3 hours: average 0.52‰
4 hours: average 0.28‰
5 hours: average 0.10‰

### Test 7b, Pinch=Fermented Rice Bran+0.5% Dextrin+10% L-Cysteine

15 participants, male 10 and 5 female, age 21 to 65, weight 42 to 125 kg with Pinch
15 minutes: average 0.96‰
1 hour: average 0.68‰
2 hours: average 0.31‰
3 hours: average 0.17‰
4 hours: average 0.03‰

### Test 8

### Test 8a

14 participants, male 9 and 5 female, age 26 to 65, weight 44 to 125 kg without Pinch
15 minutes: average 0.93‰
1 hour: average 0.78‰
2 hours: average 0.63‰
3 hours: average 0.48‰
4 hours: average 0.24‰
5 hours: average 0.07‰

Test 8b, Pinch=Fermented rice bran+5% dextrin+20% L-Cysteine 12 participants, male 8 and 4 female, age 26 to 65, weight 44 to 125 kg with Pinch
15 minutes: average 0.96‰
1 hour: average 0.75‰
2 hours: average 0.44‰
3 hours: average 0.21‰
4 hours: average 0.03‰

### Microbial Characterisation

Using Sanger DNA sequencing, it was identified that the dominant bacterial strains found in the compositions used were Bacillus subtilis spp and Bacillus coagulans spp. Other genus/species identified were Bacillus amyloliquefaciens, Bacillus velezensis, Bacillus sp MT 03, Bacillus atrophaeus, and Pediococcus pentosaceus.

### Dextrin and L-Cysteine

Dextrin is available from several suppliers. Dextrin from three different suppliers was tested and confirmed to work (data not shown). The preferred dextrin that was used herein was hydrolysed corn dextrin (e.g. Vitargo).

L-Cysteine is also available from several suppliers. L-cysteine from three different suppliers was tested and confirmed to work (data not shown). The preferred L-cysteine that was used herein was of vegetable origin.

### CONCLUSIONS

It was concluded that fermented rice bran comprising two dominant Bacillus strains, i.e. subtilis and coagulans, has a basic alcohol digesting effect of 25-30% faster than the alcohol digestion by the liver only. Additionally, the results above show that dextrin speeds up the alcohol digestion further, approximately 35-40% faster than liver digestion. A very marginal difference of added effects using 0.5 to 5% dextrin was observed. A composition of 79.5% w/w fermented rice bran (comprising at least 10,000 cfu/g of bacteria of the Bacillus genus), 0.5% w/w dextrin and 20.0% w/w L-cysteine was assessed to be the optimal mixture for digesting alcohol in the intestinal tract from a clinical perspective, having about 50% faster than liver digestion of alcohol. A combination of 0.5% w/w dextrin and 20.0% w/w L-cysteine was therefore used examples 2 and 3 below. The composition used in examples 2 and 3 therefore comprised, per dose (of 2 capsules): 73% w/w fermented rice bran (552 mg), 20% w/w L-cysteine (150 mg), 5% w/w microcrystalline cellulose (40 mg), 0.64% w/w magnesium stearate (4.8 mg), 0.5% w/w dextrin (4 mg), 0.53% w/w silicon dioxides (4 mg), 0.00012% w/w vitamin B12 (0.0009 mg) and at least 1×10⁵ cfu of bacteria of the Bacillus genus.

### Mode of Action

Without being bound to this hypothesis, the inventors believe that both dextrin and/or L-cysteine create a micro-environment on a cellular level that makes the microbial consortium, upon resuscitation in the intestinal tract, excrete an enzyme cascade targeted towards short carbon chains, e.g. ethanol/alcohol—resulting in preferential targeting/preferential use of ethanol/alcohol as a substrate.

Generally, about 80% of the intake of alcohol resides in the small intestine for quite some time before being absorbed to the blood. The targeted enzymes act only on the alcohol in the tract and break it down into carbon dioxide and water, bypassing the liver process of converting alcohol into acetaldehyde and acetic acid, i.e. hangover metabolites.

### Example 2—Dietary Supplement Comparative Trial PERA-ATX-001: Clinical Study to Assess the Impact of a Bacterial-Based Nutritional Supplement (AB001) on Ethyl Alcohol Absorption in the Intestinal Tract

### Study Summary

TABLE 1
PERA-ATX-001 study summary.

| | |
|---|---|
| Study Title | Clinical Study to Assess the Impact of a Bacterial |
| | Nutritional Supplement (AB001) on Ethyl Alcohol |
| | Absorption in the Intestinal Tract |
| Trial design | Prospective, randomized, double-blind, cross-over, |
| | placebo-controlled |
| Primary | Primary objective was to investigate the impact of |
| Objective | AB001 on alcohol uptake into the blood as compared |
| | to placebo |
| Secondary | Secondary objective was to measure the impact of |
| Objectives | AB001 on ethyl alcohol concentrations in the breath |
| | Another secondary objective was to evaluate the |
| | impact of AB001 on cognitive function 1 h after |
| | alcohol uptake |
| | Another secondary objective was the tolerability of |
| | the supplementary study intervention during the study. |
| Efficacy | plasma levels of ethyl alcohol |
| parameters | ethyl alcohol concentrations in the breath |
| | results of the number connection test |
| Patients | 24 healthy volunteers (13 male, 11 female, age: |
| | 25.4 ± 7.7 yrs, BMI: 23.6 ± 2.5 kg/m²). All subjects |
| | performed the study per protocol. |
| Efficacy | There was a significant reduction of blood alcohol |
| Results | levels by 70.3% (p < 0.005) with AB001, when compared |
| | to placebo. There was a less pronounced but also |
| | significant reduction of alcohol in the breath test |
| | 30.7% (p < 0.005), when compared to placebo. No |
| | difference in the cognitive function test between |
| | verum and placebo could be observed 60 min after |
| | alcohol ingestion (22.4 ± 7.7 s vs. 22.7 ± 5.6, n.s.). |
| Safety | The supplement uptake was well tolerated. There were |
| results | no adverse events or serious adverse events reported |
| | in this study |

### INTRODUCTION

As discussed elsewhere herein, the inventors have developed an alcohol degrading composition (AB001) to help avoid problems associated with alcohol ingestion. It is composed of naturally fermented rice bran, Bacillus subtilis and Bacillus coagulans, L-cysteine, and dextrin. It also includes magnesium stearate and calcium and potassium phosphates. The supplement comes in acid resistant capsules, HPMC, which are dissolved once reaching the duodenum. The cultures are released and settle in the upper part of the intestinal tract where they stay for about one day before being eliminated from the body through the feces. The bacterial strains included in the composition were selected to preferably and effectively metabolize ethyl alcohol into CO₂ and water, thus reducing the further absorption of alcohol from the intestinal tract. In consequence, less alcohol is expected to be absorbed by the body, and damage of organs through alcohol degradation products is expected to be diminished.

The purpose of this study was to evaluate the decrease in alcohol absorption into the blood and breath alcohol levels when drinking a defined amount of alcohol after one week of dietary supplementation with either AB001 or placebo in healthy subjects. In addition, cognitive function one hour after the alcohol uptake and the tolerability of both interventions was explored.

### Patients and Methods

### Study Population

It was planned to run the study as a prospective double-blind randomized crossover study in 24 healthy adult volunteers, which were not allowed to suffer from any disease that may have an impact on alcohol digestion or tolerability (e.g. gastrointestinal or metabolic disorders, alcohol addiction, allergies etc.).

### Study Schedules

The study was conducted in accordance with international and local ethical and scientific standards. The protocol was approved by the responsible ethical review board (Landesärztekammer Rheinland-Pfalz, Mainz, Germany), and announced to the responsible national authority (Bundesamt für Verbraucherschutz und Lebensmittelsicherheit). Prior to participation, the participants signed written informed consent. Thereafter, blood was drawn for the safety analysis and to identify potential exclusion criteria and the randomization into the two study arms took place (visit 1).

The enrolled subjects were asked to participate in two experimental procedures (visits 2 and 3) after one week each of regular administration of two capsules per day of placebo or AB001. After arrival at the study site, the subjects ate a light breakfast, and thereafter they ingested 0.3 g alcohol/kg bodyweight of a high alcoholic spirit (vodka). A breath test (Dräger Alcotest 3820, Dräger Safety AG, Lübeck, Germany) and a blood draw for measurement of alcohol in a central laboratory (Labor Augsburg, Augsburg, Germany, gaschromatography) at timepoints 0 min, 15 min, 30 min, 45 min, 60 min, 90 min, 120 min, 180 min, 240 min, 300 min, and 360 min were done. Prior to drinking the alcohol and after 60 min, the participants were asked to perform a number connection test. The time required for completion of the test was documented. The experiment was run for at least 120 min, and until no alcohol was seen in the breath test at two consecutive timepoints.

After the second experiment (visit 3), the patients were dismissed from the study.

### Statistical Analysis

The area under the curve (AUC) of blood alcohol levels over time was used to calculate the reduction in alcohol absorption by AB001. In general, this can be done in two ways:

A. The AUC can be calculated for each participant individually by employing the following formula to each timepoint Tk:

$$AUC ( 0 - 180 ) = {\sum }_{-} ( k = 0 ) \hat{} 180 ( C [ k + 1 ] - C [ k ] ) / 2 * ( [ k + 1 ] - [ k ]$$

where T[k] and T[k+1] are two consecutive timepoints, and C[k+1] and C[k] are the alcohol concentrations measured at reach respective timepoint and T[k+1]-T[k] is the time interval between the measurements. This was done for each individual and for both treatment groups. The two-sided student's T-Test was used to calculate the p-value (5% error, 80% power, type 1) for differences between the groups.

B. Alternatively, the mean±STD of the blood alcohol results for each timepoint was calculated and the area under the curve was calculated for each treatment. The contribution of each timepoint to the AUC was calculated and two-sided student's T-Test was used to calculate the p-value (5% error, 80% power, type 1) for differences between the groups.

The result of these calculations for blood alcohol were p=0.0030 (A) and p=0.0023 (B), and p=0.0463 (A) and p=0.0041 (B) respectively (see results section below).

### Results

### Protocol Deviations

There were no protocol deviations occurring in this trial. However, the sponsor informed the Investigative Site that a mix-up had occurred with the treatment packages for Subject 13, and that the correct supplement sequence was verum (box 1) followed by placebo (box 2). The same problem was reported to be potentially present for patients 21 to 24. This information was confirmed after unblinding by the respective results, and the patients were hence included with the reversed randomization sequence in the final analysis.

### Patients

As planned, 24 healthy subjects were enrolled into the study (13 men, 11 women, mean age: 25.4±7.7 yrs (range: 18 yrs.-55 yrs.), BMI: 23.6±2.5 kg/m² (range: 19.1 kg/m²-29.1 kg/m²). The list of patients and individual patient characteristics as well as blood pressure, and the amount of alcohol ingested are provided in Table 2 and Table 3.

TABLE 2
Patient characteristics.

| Patient | | age | weight | BMI | |
|---|---|---|---|---|---|
| No. | Rand. | [years] | [kg] | [kg/m²] | gender |
| 1 | P/V | 25 | 89 | 29.1 | m |
| 2 | V/P | 29 | 77 | 23.8 | m |
| 3 | P/V | 26 | 51 | 21.2 | w |
| 4 | P/V | 19 | 66 | 22.1 | w |
| 5 | V/P | 27 | 77 | 24.3 | m |
| 6 | P/V | 19 | 65 | 20.1 | m |
| 7 | P/V | 25 | 83 | 24.8 | m |
| 8 | V/P | 22 | 56 | 21.1 | w |
| 9 | V/P | 25 | 68 | 21.2 | w |
| 10 | V/P | 20 | 59 | 23.9 | w |
| 11 | P/V | 26 | 75 | 24.5 | m |
| 12 | V/P | 23 | 95 | 27.2 | m |
| 13 | P/V | 24 | 73 | 23.3 | w |
| 14 | P/V | 22 | 65 | 23.9 | w |
| 15 | P/V | 22 | 67 | 25.2 | w |
| 16 | P/V | 31 | 62 | 19.1 | m |
| 17 | V/P | 25 | 77 | 22.7 | m |
| 18 | V/P | 20 | 72 | 21.3 | m |
| 19 | V/P | 55 | 67 | 25.5 | w |
| 20 | V/P | 19 | 67 | 21.14 | m |
| 21 | P/V | 18 | 73 | 24.7 | w |
| 22 | V/P | 32 | 77 | 28 | w |
| 23 | V/P | 21 | 73 | 22.5 | m |
| 24 | P/V | 35 | 88 | 24.9 | m |
| Mean | | 25.4 | 71.8 | 23.6 | 11 |
| Std. | | 7.7 | 10.4 | 2.5 | 13 |

TABLE 3
Blood pressure, weight and amount of alcohol (32% vodka) ingested at the different visits.

| | v1 | | | v2 | | | | v3 | | | |
|---|---|---|---|---|---|---|---|---|---|---|---|
| | sys. | diast. | | sys. | diast. | | alcohol | sys. | diast. | | alcohol |
| Patient | BP | BP | weight | BP | BP | weight | uptake | BP | BP | weight | uptake |
| No. | (mmHg) | (mmHg) | (kg) | (mmHg) | (mmHg) | (kg) | mL | (mmHg) | (mmHg) | (kg) | mL |
| 1 | 112 | 56 | 90 | 112 | 65 | 90 | 85 | 117 | 67 | 91 | 85 |
| 2 | 124 | 76 | 77 | 113 | 68.5 | 77 | 72 | 112.5 | 68.5 | 77 | 72 |
| 3 | 103.5 | 66 | 50 | 121.5 | 64 | 50 | 47 | 107 | 70 | 51 | 48 |
| 4 | 132.5 | 75.5 | 66 | 121 | 65 | 66 | 62 | 126 | 65 | 68 | 63 |
| 5 | 117 | 66.5 | 78 | 116 | 66 | 78 | 73 | 115.5 | 68 | 78 | 73 |
| 6 | 120.5 | 71.5 | 66 | 120 | 68.5 | 66 | 62 | 127 | 68 | 66 | 61 |
| 7 | 134.5 | 69.5 | 83 | 133.5 | 72 | 84 | 79 | 132.5 | 74.5 | 85 | 79 |
| 8 | 112 | 70.5 | 56 | 120 | 76.5 | 56 | 52 | 98.5 | 66 | 56 | 52 |
| 9 | 104 | 71.5 | 68 | 103.5 | 76 | 67 | 63 | 102.5 | 64.5 | 67 | 63 |
| 10 | 107.5 | 63.5 | 59 | 117 | 61 | 59 | 55 | 108 | 60.5 | 60 | 56 |
| 11 | 119.5 | 68 | 75 | 135.5 | 82.5 | 77 | 72 | 129.5 | 84 | 77 | 72 |
| 12 | 127 | 66 | 95 | 123 | 64.5 | 95 | 89 | 121 | 64 | 95 | 89 |
| 13 | 120.5 | 81 | 74 | 126 | 80 | 74 | 69 | 108 | 71 | 74 | 69 |
| 14 | 146 | 99.5 | 65 | 142.5 | 98.5 | 68 | 63 | 142.5 | 95 | 67 | 62 |
| 15 | 123.5 | 72 | 67 | 118.5 | 75.5 | 66 | 62 | 128 | 74.5 | 67 | 63 |
| 16 | 104 | 64.5 | 61 | 99.5 | 62 | 62 | 58 | 103.5 | 54 | 62 | 58 |
| 17 | 129 | 71 | 71 | 137 | 73.5 | 79 | 74 | 126 | 75 | 79 | 74 |
| 18 | 124.5 | 67 | 77 | 116 | 66.5 | 72 | 68 | 105.5 | 66.5 | 72 | 68 |
| 19 | 147 | 86.5 | 66 | 148.5 | 95 | 69 | 64 | 141.5 | 88.5 | 68 | 64 |
| 20 | 139 | 69.5 | 67 | 141.5 | 79.5 | 66 | 61 | 147 | 75 | 65 | 61 |
| 21 | 118 | 64.5 | 73 | 112.5 | 63.5 | 73 | 69 | 113.5 | 55.5 | 74 | 67 |
| 22 | 91.5 | 68 | 77 | 114 | 71 | 79 | 74 | 104.5 | 63.5 | 79 | 74 |
| 23 | 121 | 66 | 73 | 132 | 67 | 76 | 72 | 120.5 | 59 | 76 | 71 |
| 24 | 124 | 74.5 | 88 | 128 | 73 | 89 | 84 | 127.5 | 64 | 89 | 83 |
| AVERAGE | 120.9 | 71.0 | 71.8 | 123.0 | 72.3 | 72.4 | 67.9 | 119.4 | 69.2 | 72.6 | 67.8 |
| STDEV | 13.5 | 8.6 | 10.6 | 12.3 | 9.6 | 10.8 | 10.3 | 13.6 | 9.6 | 10.8 | 10.1 |

### Statistical Analysis Population

All patients performed the study per protocol and were included into the safety and efficacy analysis.

### Efficacy Results

In general, the amount of alcohol ingested (0.3 g/kg body weight), which was approved by the IRB was low (47 mL to 89 mL of a spirit containing 32% of alcohol) and did not result in measurable blood alcohol concentrations with any of the two interventions in 6 subjects (25%). From the remaining participants, 4 did not show a blood alcohol concentration with verum or placebo above 0.1‰ (17% of the population tested). In the placebo experiment, no detectable breath alcohol concentration at all was seen in 3 subjects. Values with breath alcohol levels above 0.1‰ were detected in 18 subjects in this group. The highest observed alcohol concentrations were both seen in the placebo experiments and were 0.33‰ (breath) and 0.27‰ (blood), respectively (verum: 0.30‰ and 0.21‰).

### Blood Alcohol Results

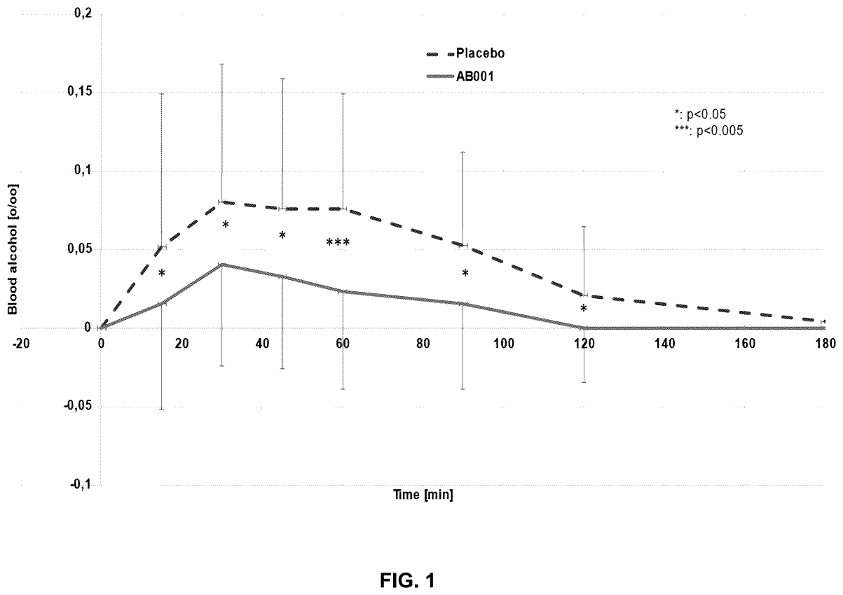

The mean blood concentrations after one week of regular nutritional supplementation with 2 capsules/day of study product or placebo are provided in FIG. 1.

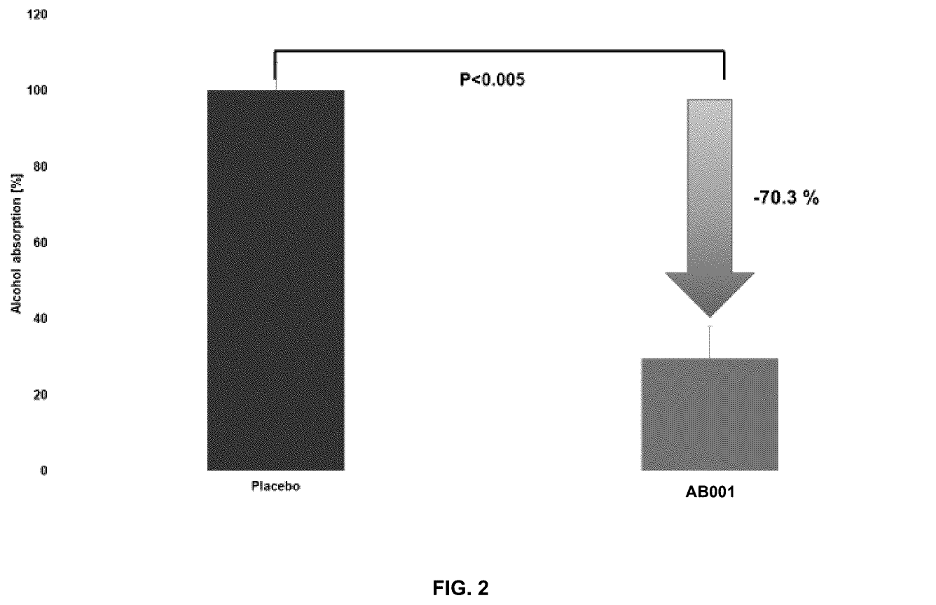

There was no measurable alcohol level in the blood detectable beyond 180 min in any of the individual experiments. The AUC_{Blood (0-180 min)} was calculated to be 8.5±0.6 o/oo*min from the placebo experiments and 2.5±0.2*min from the verum experiments (−70.3%; p<0.005, see FIG. 2).

### Breath Alcohol Results

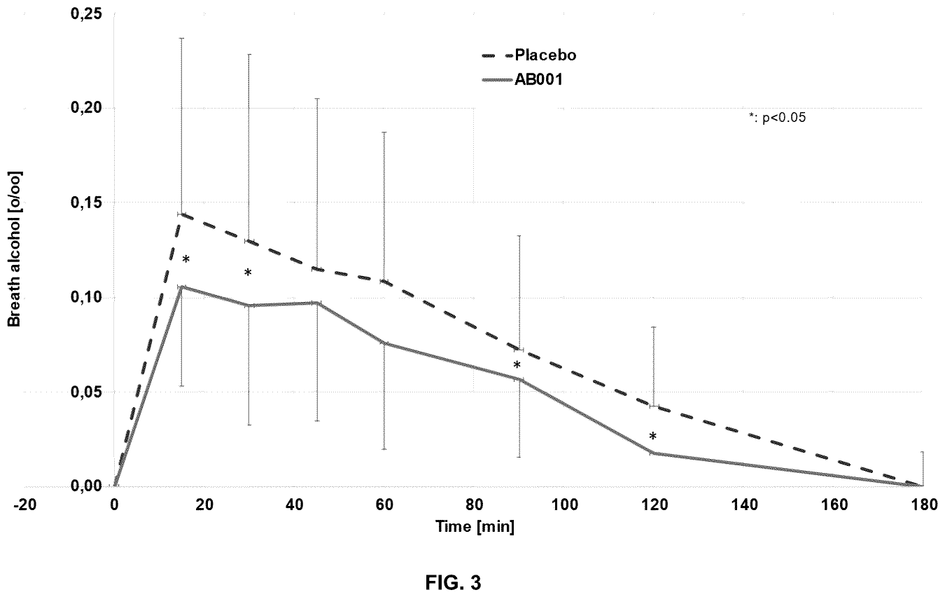

The mean breath alcohol concentrations after one week of regular nutritional supplementation with 2 capsules/day of study product or placebo and uptake of 0.3 g/kg body weight of alcohol are provided in FIG. 3.

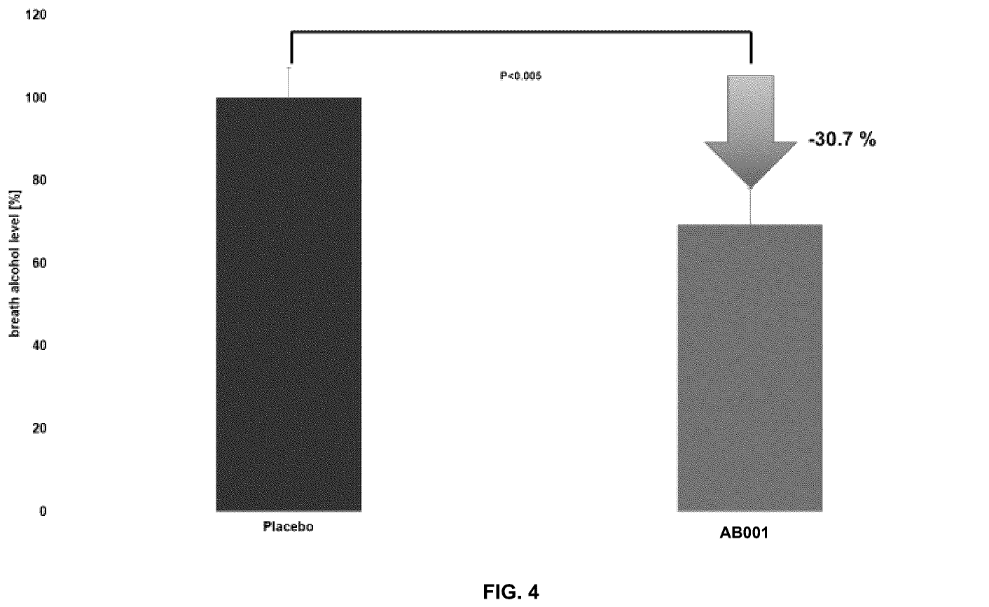

There was no measurable alcohol level in the breath detectable beyond 180 min in any of the individual experiments. The AUC_{Breath (0-180 min)} as calculated from the mean concentrations was 14.0 o/oo*mL from the placebo experiments and 9.7 o/oo*mL from the verum experiments (−30.7%, p<0.005, see FIG. 4).

### Number Connection Test

The uptake of the ingested amount of alcohol did not impact the cognitive function of the patients as assessed by measuring the time required to complete the standardized number connection test. The individual results are shown in Table 4.

TABLE 4
Time requirement for the number connection
test at baseline (0 min) and after 60 min

| | Baseline (0 min) | | 60 min | |
|---|---|---|---|---|
| Subject | AB001 | Placebo | AB001 | Placebo |
| 1 | 17 | 24 | 20 | 24 |
| 2 | 17 | 16 | 22 | 14 |
| 3 | 16 | 30 | 28 | 23 |
| 4 | 13 | 17 | 12 | 24 |
| 5 | 25 | 21 | 19 | 22 |
| 6 | 20 | 27 | 29 | 25 |
| 7 | 11 | 28 | 14 | 15 |
| 8 | 32 | 14 | 23 | 18 |
| 9 | 19 | 18 | 14 | 16 |
| 10 | 21 | 23 | 19 | 19 |
| 11 | 13 | 14 | 12 | 19 |
| 12 | 25 | 28 | 33 | 25 |
| 13 | 18 | 19 | 19 | 31 |
| 14 | 18 | 28 | 24 | 22 |
| 15 | 19 | 25 | 18 | 27 |
| 16 | 29 | 42 | 29 | 28 |
| 17 | 24 | 17 | 25 | 22 |
| 18 | 24 | 17 | 18 | 16 |
| 19 | 33 | 18 | 43 | 35 |
| 20 | 21 | 14 | 19 | 16 |
| 21 | 24 | 17 | 22 | 23 |
| 22 | 22 | 44 | 27 | 35 |
| 23 | 17 | 20 | 14 | 18 |
| 24 | 40 | 31 | 31 | 25 |
| Mean ± STD | 21.6 ± 6.8 | 23.0 ± 8.2 | 22.3 ± 7.5 | 22.6 ± 5.8 |

There was no difference in the time requirements to complete cognitive function test between verum and placebo prior to the experiment and also one hour into the experiment after the uptake, respectively.

### Safety Analysis

The nutritional supplement was well tolerated, and there were no adverse events or serious adverse events reported in this trial. It can be concluded that the nutritional supplement has had no side effects in this trial.

In addition, there were no clinically relevant deviations from normal values observed in the safety biochemistry panels taken before and after the study.

### DISCUSSION AND CONCLUSIONS

As presented herein, a substantial reduction of alcohol absorption into the blood by more than 70% was observed after one week of AB001 supplementation as compared to placebo. The reduction of measurable alcohol in the breath was also reduced significantly but to a lower extent (by ˜30%). It is well known that breath and blood alcohol have a high correlation, but the results of breath-alcohol measurement are more prone to physiological variations such as body and breath temperature, pulmonary function, and pattern of breathing prior to exhalation (Jones et al., 2000). It is therefore possible that the final alcohol content in breath is normally more driven by the alcohol that is early absorbed in the upper ingestion tract, i.e. in the buccal mucosa and in the stomach. This would explain why the measured impact was more pronounced in the blood tests than in the breath tests.

It is noted that the amount of alcohol ingested (0.3 g/kg body weight) did not lead to measurable relevant alcohol concentrations in the blood in 10 cases (42%). The amount of alcohol to be ingested in this study was defined by the IRB in the study approval process.

In conclusion, one week of regular supplementation with AB001 resulted in a substantially lower uptake of alcohol into the blood and hence into the systemic metabolism by more than 70%. Regular uptake of AB001 as a nutritional supplement may therefore help to prevent liver and other organ damage known to be associated with regular alcohol uptake and may reduce the negative medical and economical impact of social drinking on the individual and society.

### References for Example 2

Jones A W. Medicolegal Alcohol Determination—Blood— or breath alcohol concentration. Forensic Sci. Rev. 12:23-47, 2000.

### Example 3—Dietary Supplement Comparative Trial PERA-ATX-002: Prospective randomized Study to Assess the Impact of a Single Dose of a Nutritional Supplement (AB001) on Alcohol Absorption in Healthy Subjects

### Study Summary

TABLE 5
PERA-ATX-002 study summary.

| | |
|---|---|
| Study Title | Prospective randomized Study to Assess the Impact of |
| | a Single Dose of a Nutritional Supplement (AB001) on |
| | Alcohol Absorption in Healthy Subjects |
| Trial design | Prospective, randomized, double-blind, cross-over, |
| | placebo-controlled |
| Primary | Primary objective was to investigate the impact of a |
| Objective | single dose of AB001 on alcohol uptake into the blood |
| | as compared to placebo |
| Secondary | Secondary objective was to measure the impact of a |
| Objectives | single dose of AB001 on ethyl alcohol concentrations |
| | in the breath |
| | Another secondary objective was to evaluate the |
| | impact of a single dose of AB001 on cognitive |
| | function 1 h after alcohol uptake |
| | Another secondary objective was the tolerability of |
| | the supplementary study intervention during the study. |
| Efficacy | plasma levels of ethyl alcohol |
| parameters | ethyl alcohol concentrations in the breath |
| | results of the number connection test |
| Patients | 24 healthy volunteers (12 male, 12 female, age: |
| | 28.3 ± 10.8 yrs, BMI: 23.5 ± 5.7 kg/m²). All subjects |
| | performed the study per protocol. |
| Efficacy | There was a significant reduction of blood alcohol |
| Results | levels by 10.1% (p < 0.001) with AB001, when compared |
| | to placebo. There was a less pronounced but also |
| | significant reduction of alcohol in the breath test |
| | by 7.2% (p < 0.05), when compared to placebo. No |
| | difference in the cognitive function test between |
| | AB001 and placebo could be observed 60 min after |
| | alcohol ingestion (22.6 ± 8.0 s vs. 23.0 ± 11.2, n.s.). |
| Safety | The supplement uptake was well tolerated. There were |
| results | two adverse events (mild case of anemia and moderate |
| | case of headache) reported in this study, which were |
| | both classified to be “not related” to the study |
| | intervention |

As described above, the inventors have developed an alcohol degrading supplement (AB001) to help avoid problems associated with alcohol ingestion. It is composed of naturally fermented rice bran, Bacillus subtilis and Bacillus coagulans, L-Cysteine and dextrin. It also includes magnesium stearate and calcium and potassium phosphates. The supplement comes in acid resistant capsules, HPMC, which are dissolved once reaching the duodenum. The cultures are released and settle in the upper part of the intestinal tract where they stay for about one day before being eliminated from the body through the feces. The bacterial strains were selected to preferably and effectively metabolize ethyl alcohol into CO₂ and water, thus reducing the further absorption of alcohol from the intestinal tract. In consequence, less alcohol is expected to be absorbed by the body, and damage of organs through alcohol degradation products is expected to be diminished.

Example 2 provides a first randomized placebo-controlled double-blind crossover study wherein 24 healthy subjects (13 male, 11 female, age: 25.4±7.7 yrs, BMI: 23.6±2.5 kg/m²) were randomized to take 2 capsules/day of AB001 or placebo for one week prior to an alcohol exposure experiment. On the experimental day, they ingested a light breakfast and drank a moderate glass of spirit (0.3 g/kg body weight). Breath alcohol tests and blood draws for determination of blood alcohol levels were performed for up to 6 hours. Areas under the curves were calculated to determine alcohol absorption rates. A significant reduction of blood alcohol levels by 70.3% (p<0.005 vs. placebo) was seen with AB001, (breath test: −30.7%; p<0.005 vs. placebo). No difference was seen in a cognitive function test performed 60 min after alcohol ingestion (22.4±7.7 sec vs. 22.7±5.6 sec, n.s.). There were no adverse events or serious adverse events reported in this study.

The purpose of the study of Example 3 was to continue the scientific investigation of the performance of AB001 and to answer the following questions:
1. What result can be obtained if only a single dose of AB001 is ingested directly prior to the alcohol uptake in contrast to the 7-day supplementation in the previous study?
2. Is the observed inhibitory effect also maintained at a higher dose of alcohol?

### Patients and Methods

### Study Population

It was planned to run the study as a prospective double-blind randomized crossover study in 24 healthy adult volunteers, which were not allowed to suffer from any disease that may have an impact on alcohol digestion or tolerability (e.g. gastrointestinal or metabolic disorders, alcohol addiction, allergies etc.; for detailed inclusion and exclusion criteria, please refer to the study protocol).

### Study Schedules

The study was conducted in accordance with international and local ethical and scientific standards. The protocol was approved by the responsible ethical review board (Landesärztekammer Rheinland-Pfalz, Mainz, Germany) and announced to the responsible national authority (Bundesamt for Verbraucherschutz und Lebensmittelsicherheit).

Prior to participation, the participants signed written informed consent. Thereafter, blood was drawn for the safety analysis and to identify potential exclusion criteria and the randomization into the two study arms took place (visit 0).

The enrolled subjects were asked to participate in two experimental procedures (visits 1 and 2). After arrival at the study site in the morning after an overnight fast, they were randomized to receive either placebo or the AB001 supplement. One hour after ingesting the investigational product the participant drank a first shot of a high alcoholic spirit (vodka; 0.3 g/kg body weight, timepoint 0 min) and consumed a light breakfast with rolls, ham or jam and with tea or coffee. Thereafter, they drank a second glass of alcohol (vodka; 0.3 g/kg bodyweight, timepoint 30 min). Blood was drawn for determination of plasma alcohol concentrations and breathalyzer assessments were be made for determination of breath alcohol concentrations after 0, 15, 30, 45, 60, 75, 90, 120, 180, 240, 300, 360, and 420 min. The participants stayed at the study site for the whole time. A standardized lunch was served after 4 hours. In addition, the participants performed a cognitive function test (number connection test, NCT-A) at time-points 0 min and 60 min. Information regarding adverse events was documented. If no alcohol was detectable in the breath at two consecutive timepoints after the second alcohol uptake, the experiment was prematurely terminated.

The second experiment was performed 3 to 5 days later following the same experimental protocol. After the second experiment (visit 2), the patients were dismissed from the study.

### Statistical Analysis

The area under the curve (AUC) of blood alcohol levels over time were used to calculate the reduction in alcohol absorption by AB001. In general, this can be done by two ways:

A. The AUC can be calculated for each participant individually by employing the following formula to each timepoint Tk:

$$AUC ( 0 - 180 ) = {\sum }_{-} ( k = 0 ) \hat{} 180 ( C [ k + 1 ] - C [ k ] ) / 2 * ( [ k + 1 ] - [ k ]$$

where T[k] and T[k+1] are two consecutive timepoints, and C[k+1] and C[k] are the alcohol concentrations measured at reach respective timepoint and T[k+1]-T[k] is the time interval between the measurements. This was done for each individual and for both treatment groups. The two-sided student's T-Test was used to calculate the p-value (5% error, 80% power, type 1) for differences between the groups.
B. Alternatively, the mean±STD of the blood alcohol results for each timepoint was calculated and the area under the curve was calculated for each treatment. The contribution of each timepoint to the AUC was calculated and two-sided student's T-Test was used to calculate the p-value (5% error, 80% power, type 1) for differences between the groups.

The result of these calculation for blood alcohol were p=0.014 (A) and p=0.000019 (B), and p=0.0448 (A) and p=0.000074 (B) for breath alcohol, respectively (see result section below).

### Results

### Protocol Deviations

There were no protocol deviations occurring in this trial.

### Patients

As planned, 24 healthy subjects were enrolled into the study (12 men, 12 women, mean age: 28.3±10.8 yrs (range: 20 yrs.-56 yrs.), BMI: 23.5±5.7 kg/m² (range: 16.9 kg/m²-31.1 kg/m²). The list of patients and individual patient characteristics as well as blood pressure, and the amount of alcohol ingested are provided in Table 6 and Table 7.

TABLE 6
patient characteristics.

| Patient | | age | weight | BMI | |
|---|---|---|---|---|---|
| No. | Rand. | [years] | [kg] | [kg/m²] | gender |
| 1 | V/P | 23 | 63 | 23.71 | F |
| 2 | P/V | 23 | 66 | 24.24 | F |
| 3 | P/V | 32 | 62 | 19.14 | M |
| 4 | P/V | 25 | 74 | 23.89 | F |
| 5 | P/V | 29 | 73 | 23.04 | M |
| 6 | P/V | 27 | 50 | 20.81 | F |
| 7 | V/P | 56 | 66 | 25.15 | F |
| 8 | P/V | 20 | 66 | 20.37 | M |
| 9 | P/V | 30 | 110 | 31.12 | M |
| 10 | V/P | 21 | 69 | 21.78 | M |
| 11 | P/V | 23 | 55 | 16.94 | F |
| 12 | P/V | 22 | 58 | 23.53 | F |
| 13 | V/P | 31 | 82 | 25.31 | M |
| 14 | V/P | 26 | 86.2 | 25.74 | F |
| 15 | P/V | 55 | 104.3 | 28.89 | M |
| 16 | P/V | 27 | 82 | 26.78 | M |
| 17 | V/P | 20 | 65 | 21.72 | F |
| 18 | P/V | 28 | 49 | 18.9 | F |
| 19 | V/P | 22 | 75 | 23.15 | M |
| 20 | V/P | 23 | 61 | 21.61 | F |
| 21 | V/P | 26 | 86 | 26.84 | M |
| 22 | V/P | 33 | 75 | 27.55 | F |
| 23 | P/V | 36 | 85 | 24.05 | M |
| 24 | P/V | 21 | 69 | 20.16 | M |
| Mean | | 28.3 | 72.1 | 23.5 | 12 |
| Std. | | 10.8 | 20.6 | 5.7 | 12 |

TABLE 7
Blood pressure, pulse, and amount of alcohol (32% vodka) ingested at the different visits.

| | v1 | | | v2 | | | | v3 | | | |
|---|---|---|---|---|---|---|---|---|---|---|---|
| | sys. | diast. | pulse | sys. | diast. | pulse | Alcohol | sys. | diast. | pulse | Alcohol |
| Patient | BP | BP | (beats/ | BP | BP | (beats/ | uptake | BP | BP | (beats/ | uptake |
| No. | (mmHg) | (mmHg) | min) | (mmHg) | (mmHg) | min) | mL | (mmHg) | (mmHg) | min) | mL |
| 1 | 106 | 79 | 96 | 102 | 72 | 93 | 58 | 111 | 85 | 82 | 55.13 |
| 2 | 139 | 104 | 82 | 133 | 98 | 75 | 61 | 126 | 90 | 76 | 61.41 |
| 3 | 97 | 65 | 67 | 105 | 65 | 75 | 58 | 105 | 65 | 57 | 57.09 |
| 4 | 114 | 75 | 68 | 110 | 76 | 69 | 70 | 116 | 85 | 66 | 70.5 |
| 5 | 127 | 79 | 83 | 129 | 83 | 83 | 68 | 109 | 86 | 99 | 67.59 |
| 6 | 110 | 82 | 76 | 108 | 73 | 71 | 47 | 114 | 75 | 63 | 46.88 |
| 7 | 139 | 79 | 81 | 135 | 78 | 79 | 62 | 129 | 83 | 73 | 58.03 |
| 8 | 111 | 80 | 81 | 121 | 71 | 72 | 62 | 116 | 72 | 70 | 61.88 |
| 9 | 126 | 79 | 80 | 115 | 71 | 79 | 104 | 123 | 83 | 81 | 102.84 |
| 10 | 138 | 83 | 83 | 126 | 80 | 91 | 64 | 122 | 68 | 70 | 65.91 |
| 11 | 106 | 74 | 75 | 107 | 73 | 70 | 52 | 106 | 76 | 74 | 52.22 |
| 12 | 125 | 83 | 80 | 123 | 75 | 77 | 54 | 106 | 66 | 71 | 54.47 |
| 13 | 124 | 85 | 114 | 117 | 77 | 109 | 77 | 116 | 81 | 89 | 78.84 |
| 14 | 119 | 85 | 61 | 117 | 79 | 64 | 79 | 132 | 80 | 60 | 81.47 |
| 15 | 129 | 80 | 55 | 128 | 76 | 53 | 98 | 128 | 86 | 58 | 99.56 |
| 16 | 149 | 93 | 92 | 145 | 89 | 86 | 77 | 139 | 87 | 72 | 77.81 |
| 17 | 122 | 88 | 80 | 129 | 78 | 76 | 61 | 113 | 69 | 82 | 60.94 |
| 18 | 108 | 73 | 69 | 107 | 87 | 65 | 48 | 96 | 63 | 74 | 48.66 |
| 19 | 134 | 74 | 72 | 130 | 73 | 68 | 73 | 131 | 73 | 60 | 71.44 |
| 20 | 136 | 89 | 109 | 133 | 88 | 105 | 57 | 116 | 75 | 86 | 57.75 |
| 21 | 122 | 75 | 69 | 120 | 72 | 63 | 84 | 120 | 76 | 77 | 83.44 |
| 22 | 111 | 79 | 69 | 111 | 80 | 69 | 72 | 106 | 75 | 81 | 73.59 |
| 23 | 116 | 78 | 96 | 112 | 70 | 96 | 82 | 117 | 70 | 50 | 83.44 |
| 24 | 134 | 77 | 66 | 131 | 72 | 54 | 66 | 125 | 71 | 57 | 66.19 |
| AVERAGE | 123 | 81 | 79 | 121 | 77 | 77 | 68 | 118 | 77 | 72 | 68 |
| STDEV | 13 | 8 | 14 | 11 | 7 | 14 | 14 | 10 | 8 | 12 | 15 |

### Statistical Analysis Population

All patients performed the study per protocol and were included into the safety and efficacy analysis.

### Efficacy Results

In general, the amount of alcohol ingested (2×0.3 g/kg body weight), which was approved by the IRB resulted in measurable blood alcohol levels in all subjects. The highest observed alcohol concentrations were both seen in the placebo experiments and were 0.88 o/oo (breath) and 0.91 o/oo (blood), respectively (AB001: 0.77 o/oo and 0.72 o/oo).

### Blood Alcohol Results

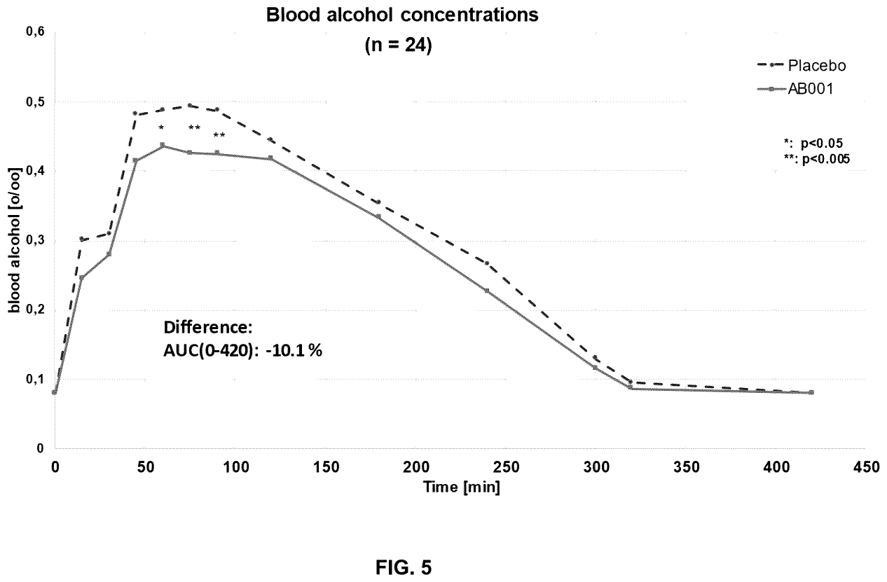

The mean blood concentrations after nutritional supplementation with 2 capsules of study product or placebo one hour before the experiment are provided in FIG. 5.

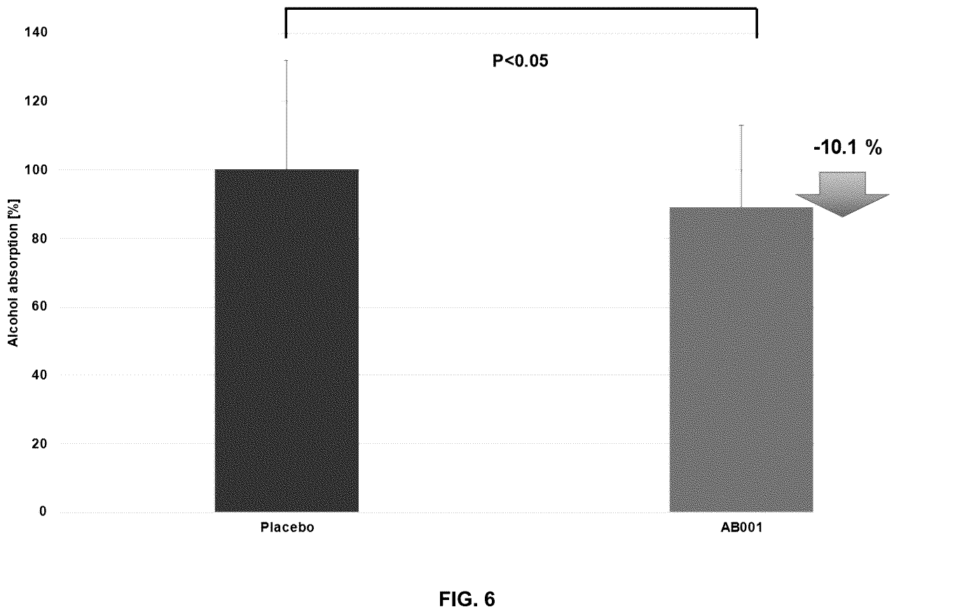

The AUC_{Blood(0-420 min)} was calculated to be 116±32 o/oo*min from the placebo experiments and 104±24 0/00*min from the AB001 experiments (−10.1%; p<0.05, see FIG. 6).

### Breath Alcohol Results

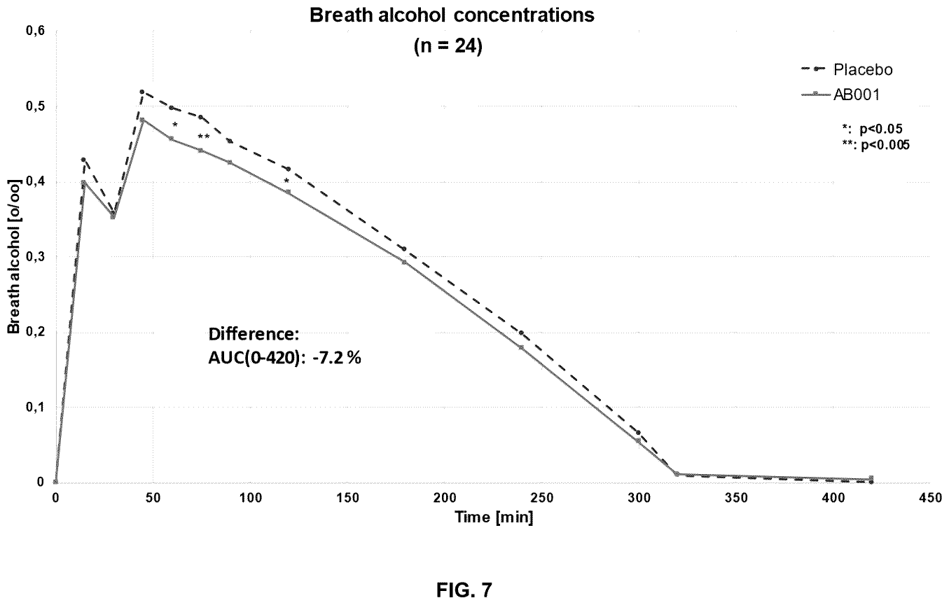

The mean breath alcohol concentrations after nutritional supplementation with 2 capsules of study product or placebo one hour before the experiment are provided in FIG. 7.

The AUC_{Breath(0-420 min)} as calculated from the mean concentrations was 98±29 o/oo*min from the placebo experiments and 91±25 o/oo*min from the AB001 experiments (−7.2%, p<0.05).

### Number Connection Test

The uptake of the ingested amount of alcohol did not impact the cognitive function of the patients as assessed by measuring the time required to complete the standardized number connection test. The individual results are shown in Table 8.

TABLE 8
Time requirement for the number connection
test at baseline (0 min) and after 60 min.

| | Baseline (0 min) | | 60 min | |
|---|---|---|---|---|
| Subject | AB001 | Placebo | AB001 | Placebo |
| 1 | 13 | 25 | 15 | 23 |
| 2 | 16 | 18 | 21 | 18 |
| 3 | 20 | 27 | 28 | 31 |
| 4 | 19 | 16 | 22 | 18 |
| 5 | 29 | 20 | 26 | 13 |
| 6 | 20 | 14 | 14 | 17 |
| 7 | 37 | 23 | 35 | 33 |
| 8 | 18 | 16 | 23 | 27 |
| 9 | 19 | 21 | 17 | 20 |
| 10 | 21 | 11 | 19 | 18 |
| 11 | 15 | 21 | 27 | 21 |
| 12 | 17 | 13 | 27 | 22 |
| 13 | 14 | 12 | 12 | 19 |
| 14 | 13 | 14 | 18 | 14 |
| 15 | 22 | 25 | 28 | 27 |
| 16 | 11 | 11 | 12 | 11 |
| 17 | 13 | 26 | 13 | 15 |
| 18 | 42 | 29 | 37 | 64 |
| 19 | 15 | 16 | 21 | 15 |
| 20 | 25 | 18 | 21 | 19 |
| 21 | 13 | 21 | 14 | 19 |
| 22 | 26 | 30 | 31 | 35 |
| 23 | 24 | 24 | 39 | 37 |
| 24 | 12 | 17 | 14 | 17 |
| Mean ± STD | 19.8 ± 7.8 | 19.5 ± 5.7 | 22.6 ± 8.0 | 23.0 ± 11.2 |

There was a minor impairment in the performance of the individual subject after uptake of the alcohol, which was statistically significant in the AB001 experiment (p<0.05) and almost significant in the placebo experiment (p=0.057).

### Safety Analysis

The nutritional supplements were well tolerated. There were two adverse events were reported in this trial.

One case of mild anemia was reported for subject 6 (female, 27 years) at screening and at visit 3 prior to the uptake of AB001. Mild anemia was diagnosed based on the following laboratory results (screening/visit 3-reference range):
erythrocytes: 3.74/3.64 Mio/μL (reference range: 3.9-5.2 Mio/μL)
hemoglobin: 11/2/11.0 g/dL (reference range: 12-16 g/dL
hematokrit: 34/33% (reference range: 36%-46%)

At screening the investigator ranked these results as “not clinically significant” and the subject could be included. It was recommended to the subject to follow-up on this condition with her family doctor after the end of the trial. This event was classified to be “not related” to the study intervention by the study investigator.

One case of moderate headache (migraine) was reported for subject 9 (male, 30 years) at visit 3 prior to uptake of AB001. The subject reported this to be a known ongoing event, which in the actual case was treated by uptake of 200 mg ibuprofen. This event was classified to be “not related” to the study intervention by the study investigator.

In addition, there were no serious adverse events reported from this trial.

It can be concluded that the uptake of the nutritional supplement was not associated with any side effects in this trial. In addition, there were no other clinically relevant deviations from normal values observed in the safety biochemistry panels taken before and after the study.

### DISCUSSION AND CONCLUSIONS

In Example 2 (PERA-ATX-001), a substantial reduction of alcohol absorption into the blood by more than 70% was observed after one week of AB001 supplementation as compared to placebo, when drinking 0.3 g/kg of alcohol after a light breakfast. The reduction of measurable alcohol in the breath was also reduced significantly but to a lower extent (by ˜30%).

In this example, it was observed that uptake of a single dose of AB001, taken only one hour prior to drinking 2×0.3 g/kg of alcohol with 30 min time interval between the shots, and during which a light meal was ingested, was also effective at reducing alcohol in the blood and breath. Specifically, the amount of alcohol in the blood was significantly reduced by about 10% and breath alcohol was significantly reduced by 7% with AB001 as compared to placebo.

In conclusion, uptake of a single dose of AB001 within 1 h prior to drinking reduces the amount of alcohol absorption in the intestinal tract leading to a significant reduction of alcohol in the blood and breath alcohol by about 10% and 7% respectively.

### Example 4—In Vivo Alcohol Study

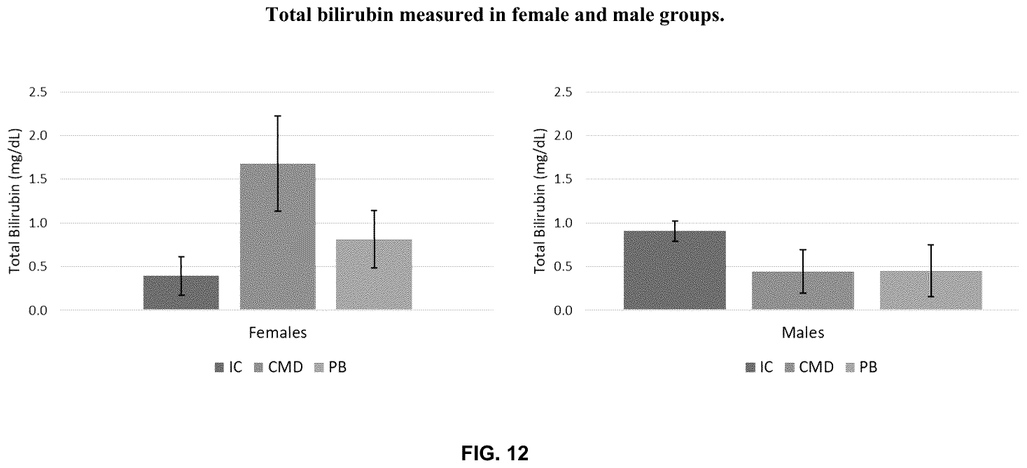

An in vivo study was performed as set out below. It demonstrated the efficiency of the fermented rice bran and the natural health beneficial Bacillus spp. using the composition described in Examples 2 and 3 (also referred to as AB001 herein). The study proved that the consumption of AB001 decreases the production of bilirubin in plasma for males during and after 10% alcohol consumption and high-fat-high-carb diet. For female groups, when out-of-range values are excluded from the raw data, it is shown that for CMD (placebo group), bilirubin increases 3.7 times (Av. 1.68 mg/dl), whereas it increases 1.8 times (Av. 0.82 mg/dl) for the PB (probiotic group) compared to IC (control group) (Av. 0.46 mg/dl), i.e. there is prevention of bilirubin increase compared to CMD. Using similar analysis, for male groups, when out-of-range values are excluded from the raw data, it is shown that CMD and PB have reduced bilirubin (to 0.44 to 0.45) respectively compared to IC's 0.91 mg/dl. This indicates that AB001 consumption is not detrimental to bilirubin levels. See FIG. 12.

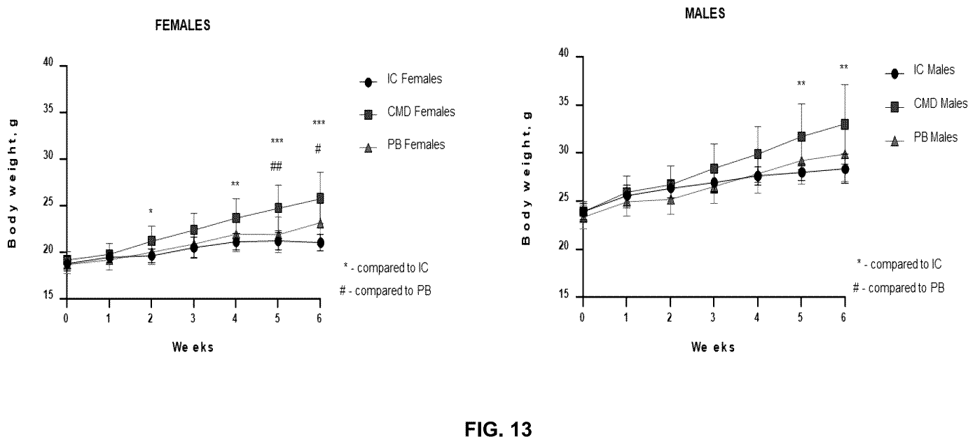

The in vivo study also demonstrated that AB001 (i.e. the composition in Examples 2 and 3) also prevents an excessive weight gain in animals that were exposed to high fat and high carbohydrate diet (western diet) (FIG. 13).

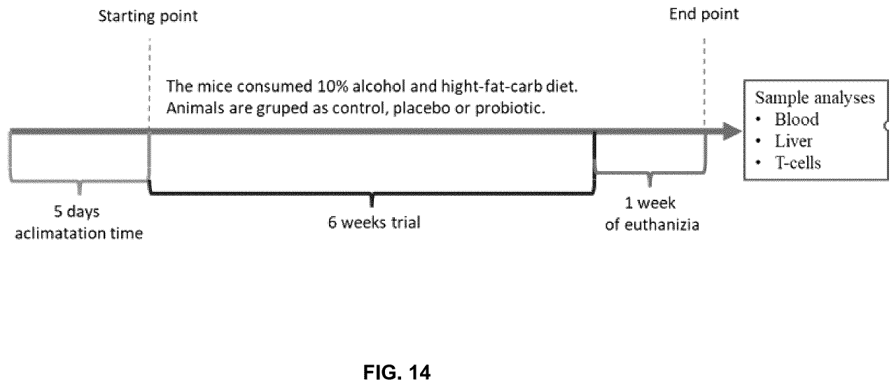

An overview of the experimental design for the in vivo alcohol study is shown in FIG. 14. The animal study was developed using 90 mice (C57BL/6JRj; Javier Labs; France) divided by gender, 8 weeks old at arrival to the experimental facility and starting of acclimation (5 days). The mice were divided and feed as listed below:
1) Intact group drank water and fed with standard rodent diet (standard rodent pelleted feed; Lactamin; Vadstena; Sweden). This group is referred to as the “IC” group herein.
2) Placebo group drank a solution of 10% ethanol in water and fed with 12% maltodextrin mixed with the western high-fat high-carb diet (D12492M diet, Research Diets, New Brunswick, NJ 08901, USA). This group is referred to as the “CMD” group herein.
3) DFM product, drank a solution of 10% ethanol, and fed with 12% of DFM product mixed with western high-fat high-carb diet (D12492M diet, Research Diets, New Brunswick, NJ 08901, USA). This group is referred to as the “PB” group herein. DFM as used herein refers to the composition of the invention, in this case AB001, as described in Examples 2 and 3.

During six weeks the mice had a cycle of 12 hours day/night. Every 5 individuals were kept in IVC cages with beta chip bedding. The drinking solutions were supplied in bottles (ad libitum) during the adaptation period and during the study. Animal body weight, feed and water consumption were measured weekly. The difference on the body weight, feed and water consumption was calculated at the beginning and final experimental day.

Blood and tissue samples were collected from anesthetized animals. Liver and spleen were collected after post-mortem with intracardial injection of pentobarbital (Allfatal, Omnidea). Plasma from the blood was collected to measure the bilirubin level. Liver left lateral lobe were stored for 24 hours after sampling in 10% neutral formalin solution for morphological analysis. For the fat content on the liver, the samples were stored at −80° C.

Statistical analysis was calculated using Prism, version 9.1.0 (GraphPad Software, Inc, San Diego, CA, USA) to distinguish if significant differences between samples were found or observed. It was considered statistically different between groups if p s 0.05.

### ABBREVIATIONS

TABLE 9
abbreviations used in Example 2 and 3.

| | |
|---|---|
| ADR | Adverse drug reaction |
| AE | Adverse event |
| ALAT | Alanin-Aminotransferase |
| AMG | Arzneimittelgesetz (“German Drug Law“) |
| ASAT | Aspartat-Aminotransferase |
| β-HCG | Humanes Choriongonadotropin, β-Kette |
| BMI | Body Mass Index |
| CRF | Case Report Form |
| CRO | Contract Research Organization |
| d | day |
| FPG | Fasting plasma glucose |
| FPI | Fasting plasma insulin |
| Hb | Hemoglobin |
| HbA_{1c} | Hemoglobin A_{1c} |
| hs CRP | high sensitivity C-reactive protein |
| HDL | High Density Lipoprotein |
| HOMA | Homeostatic Model Assessment |
| IEC | Independent Ethics Committee |
| ikfe | Institute for Clinical Research and Development GmbH |
| LDL | Low Density Lipoprotein |
| mg | Milligramme |
| min | Minutes |
| oxLDL | Oxidized Low Density Lipoprotein |
| SAE | Serious adverse event |
| T | Time |
| TM | Trade Mark |
| TSH | Thyroid stimulating Hormone |
| w | Week |

The reader's attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent, or similar purpose, unless expressly stated otherwise. Thus, unless expressly stated otherwise, each feature disclosed is one example only of a generic series of equivalent or similar features.

The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

1. An alcohol degrading composition comprising one or more bacterial species of the Bacillus genus, rice bran, L-cysteine and a high molecular weight low osmolality carbohydrate.

2. The composition of claim 1, wherein the one or more bacterial species of the Bacillus genus is selected from at least one of: B. subtilis and B. coagulans.

3. (canceled)

4. The composition of claim 2, wherein the B. subtilis species is selected from the group consisting of: Bacillus subtilis strain DFM 0326 (LMG P-32899) and Bacillus subtilis strain DFM 1015 (LMG P-32900).

5. The composition of claim 1, wherein the high molecular weight low osmolality carbohydrate is dextrin.

6. The composition of a claim 1, wherein the alcohol is ethyl alcohol.

7. The composition of a claim 1, wherein the composition further comprises one or more bacterial species selected from the group consisting of: Bacillus amyloliquefaciens, Bacillus velezensis, Bacillus sp MT 03, Bacillus atrophaeus, and Pediococcus pentosaceus.

8. The composition of m claim 1, wherein the composition comprises at least about 10% w/w of L-cysteine.

9. The composition of m claim 8, wherein the composition comprises at least about 10,000 cfu/g of bacteria of the Bacillus genus.

10. The composition of claim 9, wherein the composition comprises at least about 67% w/w of rice bran.

11. The composition of claim 10, wherein the composition comprises at least about 0.5% w/w of high molecular weight low osmolality carbohydrate.

12. The composition of claim 1, wherein the composition further comprises one or more of: vitamin B12, a fatty acid magnesium salt, calcium phosphate, potassium phosphate, silicon dioxide and cellulose.

13. The composition of claim 1, wherein the composition is formulated as an acid resistant tablet or capsule.

14. The composition of claim 13, wherein the acid resistant tablet or capsule comprises a film coating, wherein the film coating comprises hydroxypropyl methylcellulose (HPMC).

15. The composition of a claim 1, wherein the one or more bacterial species of the Bacillus genus is not genetically modified.

16.-27. (canceled)

28. The composition according to claim 1 for use in degrading ingested alcohol in a subject.

29. The composition according to claim 1 for use in preventing and/or treating alcohol-induced damage in a liver and/or pancreas of a subject resulting from ingested alcohol.

30.-32. (canceled)

33. The composition for use according to claim 28, wherein the composition is for administration before alcohol ingestion.

34.-39. (canceled)

40. The composition of claim 2, wherein the B. coagulans species is Bacillus coagulans strain DFM 0705 (LMG P-32921).

41. The composition of claim 12, wherein the fatty acid magnesium salt is magnesium stearate.
